SEQUENCE LISTING (13 sequences)

SEQ ID NO: 1 - skipped

SEQ ID NO: 2 - protein, 5 aa, synthetic construct
GGGGS

SEQ ID NO: 3 - protein, 7 aa, synthetic construct
SGGSGGS

SEQ ID NO: 4 - protein, 8 aa, synthetic construct
GGGGSGGS

SEQ ID NO: 5 - protein, 9 aa, synthetic construct
GGGGSGGGS

SEQ ID NO: 6 - protein, 10 aa, synthetic construct
GGGGSGGGGS

SEQ ID NO: 7 - protein, 15 aa, synthetic construct
GGGGSGGGGSGGGGS

SEQ ID NO: 8 - protein, 18 aa, synthetic construct
GGGGSGGGGSGGGGSGGS

SEQ ID NO: 9 - protein, 20 aa, synthetic construct
GGGGSGGGGSGGGGSGGGGS

SEQ ID NO: 10 - protein, 25 aa, synthetic construct
GGGGSGGGGSGGGGSGGGGSGGGGS

SEQ ID NO: 11 - protein, 30 aa, synthetic construct
GGGGSGGGGSGGGGSGGGGSGGGGSGGGGS

SEQ ID NO: 12 - protein, 35 aa, synthetic construct
GGGGSGGGGSGGGGSGGGGSGGGGSGGGGSGGGGS

SEQ ID NO: 13 - protein, 40 aa, synthetic construct
GGGGSGGGGSGGGGSGGGGSGGGGSGGGGSGGGGSGGGGS

(19) United States
(12) Patent Application Publication
(10) Pub. No.: US 2025/0381150 A1
(43) Pub. Date: Dec. 18, 2025

(54) LIPID NANOPARTICLES

(71) Applicant: Genzyme Corporation, Cambridge, MA (US)

(72) Inventors: Zhaohua Richard Huang, Braintree, MA (US); Sheryl Wang, Cambridge, MA (US)

(73) Assignee: Genzyme Corporation, Cambridge, MA (US)

(21) Appl. No.: 19/073,323

(22) Filed: Mar. 7, 2025

Related U.S. Application Data
(60) Provisional application No. 63/563,003, filed on Mar. 8, 2024.

(51) Int. Cl. A61K 9/51; A61K 47/68; C12N 15/88

(52) U.S. Cl. CPC A61K 9/5146; A61K 9/5123; A61K 47/6849; C12N 15/88

(57) ABSTRACT

Provided are lipid nanoparticles, compositions, and methods of making and using the same. The lipid nanoparticles contain ionizable lipids, structural lipids, PEG lipids and specific amounts of helper lipids. The lipid nanoparticles may further contain a cell targeting group coupled to a PEG lipid. The lipid nanoparticles may carry a cargo, e g., a mRNA. The lipid nanoparticles may be used for transfection of cells, e.g., immune cells or hematopoietic stem cells.

### RELATED APPLICATION

This application claims priority under 35 U.S.C. § 119(e) to U.S. Provisional Application No. 63/563,003, filed Mar. 8, 2024, which is incorporated herein by reference in its entirety.

### REFERENCE TO AN ELECTRONIC SEQUENCE LISTING

The contents of the electronic sequence listing (G069670002US01-SEQ-ACZ.xml; Size: 11,728 bytes; and Date of Creation: Mar. 17, 2025) are herein incorporated by reference in its entirety.

### FIELD

The disclosure provides lipid nanoparticles for the delivery of nucleic acids to hematopoietic stem cells and immune cells, methods of making and use.

### BACKGROUND

There exists a need for safe and effective in vivo methods for targeted expression of immune cell function enhancing polypeptides in immune cells and targeted gene editing in hematopoietic stem cells (HSC).

### SUMMARY

Provided are lipid nanoparticles for targeted delivery of nucleic acids into target cells, the lipid nanoparticles comprising an ionizable lipid, a structural lipid, a helper lipid, a PEG lipid, and a cell-targeting group, wherein the helper lipid is present in a range of about 16 mol % to about 40 mol %.

In some aspects, the ionizable lipid comprises a structure of Formula I

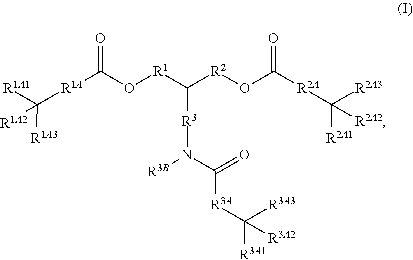

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀C(O)R^{a2};
R^{a1} and R^{a2} are each independently C₁₋₂₀ alkyl or C₁₋₂₀ alkenyl; R^{3B} is

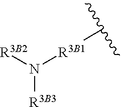

R^{3B1} is C₁₋₆ alkylene; and
R^{3B2} and R^{3B3} are each independently H or C₁₋₆ alkyl.

In some aspects, the ionizable lipid comprises a structure of Formula II (Lipid 15)

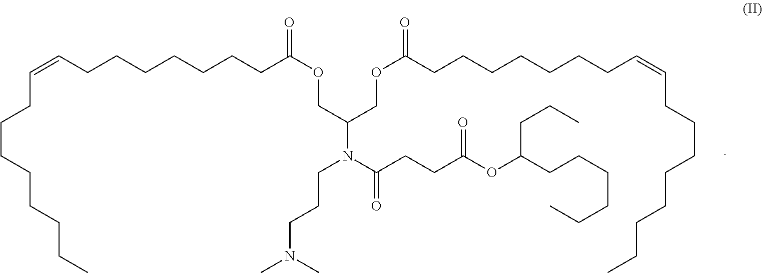 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In some aspects, the ionizable lipid comprises a structure of Formula III (KC2)

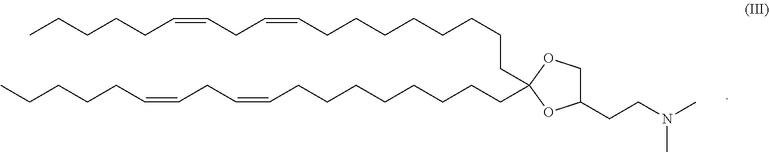 [SMILES: CCCCC/C=C\C/C=C\CCCCCCCCC1(CCCCCCCC/C=C\C/C=C\CCCCC)OCC(CCN(C)C)O1]

In some aspects, the ionizable lipid comprises a structure of Formula IV (KC3)

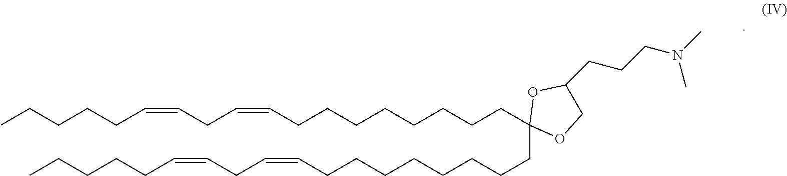 [SMILES: CCCCC/C=C\C/C=C\CCCCCCCCC1(CCCCCCCC/C=C\C/C=C\CCCCC)OCC(CCCN(C)C)O1]

In some aspects, the ionizable lipid comprises a structure of Formula V:

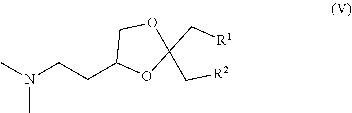

wherein R¹ and R² are each independently a lipid.

In some aspects, the ionizable lipid comprises a structure of Formula VI:

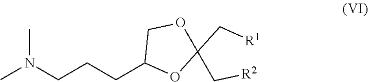

wherein R¹ and R² are each independently a lipid.

In some embodiments, where the ionizable lipid comprises a structure of Formula V or Formula VI, R¹ and R² are each independently substituted or unsubstituted alkyl, substituted or unsubstituted alkenyl. In some embodiments, where the ionizable lipid comprises a structure of Formula V or Formula VI, R¹ and R² are the same lipid. In some embodiments, where the ionizable lipid comprises a structure of Formula V or Formula VI, R¹ and R² are different lipids. In some embodiments, the lipid is a hydrocarbon (e.g., substituted or unsubstituted, saturated or unsaturated, branched or unbranched hydrocarbon). A hydrocarbon may be an alkane, alkene, or alkyne. In some embodiments, the hydrocarbon chain is saturated or unsaturated. In certain embodiments, an unsaturated hydrocarbon chain comprises at least one, at least one two, at least one three, at least one four, at least one five, or at least one six carbon-carbon double bonds (e.g., cis double bonds and/or trans double bonds). In some embodiments, the lipid is substituted or unsubstituted C₇₋₃₆ alkyl. In certain embodiments, a lipid is substituted or unsubstituted C₇₋₃₆ alkenyl. In certain embodiments, the lipid is unsubstituted C₇₋₃₆ alkyl or unsubstituted C₇₋₃₆ alkenyl. In certain embodiments, a hydrocarbon is substituted with alkyl, alkenyl, alkynyl, amino, alkylamino, dialkylamino, trialkylamino, hydroxyl, alkoxy, carbonyl, halogen, aryl, heterocyclic, aromatic heterocyclic, cyano, amide, carbamoyl, carboxylic acid, ester, thioether, alkylthioether, thiol, ureido, or a combination thereof. Each of these groups may in turn be substituted. In some embodiments, a hydrocarbon is substituted with one, two, three, four, five, six, seven, eight, nine, ten, or more than ten substituents.

In some aspects, the lipid nanoparticle further comprises a cargo.

In some aspects, the helper lipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.

In some aspects, the helper lipid is DSPC.

In some aspects, the structural lipid is selected from the group consisting of cholesterol, fecosterol, β-sitosterol, ergosterol, campesterol, stigmasterol, stigmastanol, and brassicasterol.

In some aspects, the PEG lipid is selected from the group consisting of distearoylglycerol-PEG (DSG-PEG), distearoyl-phosphatidylethanolamine-PEG (DSPE-PEG), dimyrstoyl-phosphatidylethanolamine-PEG (DMPE-PEG), distearoyl-glycero-phosphoglycerol-PEG (DSPG-PEG), dimyristoyl-glycerol-PEG (DMG-PEG), dipalmitoyl-phosphatidylethanolamine-PEG (DPPE-PEG), dipalmitoyl-glycerol-PEG (DPG-PEG), and ceramide-PEG.

In some aspects the ionizable lipid is present in a range of about 30 mol % to about 70 mol %.

In some aspects, the ionizable lipid is present in a range of about 40 mol % to about 60 mol %.

In some aspects, the ionizable lipid is present in a range of about 48 mol % to about 50 mol %.

In some aspects, the structural lipid is present in a range of about 20 mol % to about 50 mol %.

In some aspects, the structural lipid is present in a range of about 30 mol % to about 40 mol %.

In some aspects, the structural lipid is cholesterol.

In some aspects, cholesterol is present in a range of about 25 mol % to about 40 mol %.

In some aspects, the PEG lipid is present in a range of about 1 mol % to about 4 mol %.

In some aspects, the PEG lipid is DPG-PEG or DSPE-PEG.

In some aspects, the PEG in DPG-PEG has a molecular weight of about 2000 daltons (DPG-PEG2K). In some aspects, the PEG in DSPE-PEG has a molecular weight of about 2000 daltons (DSPE-PEG2K) or about 3400 daltons (DSPE-PEG3.4K).

In some aspects, DPG-PEG2K, DSPE-PEG2K, or DSPE-PEG3.4K is present in a range of about 1 mol % to about 2 mol %.

In some aspects, Lipid 15 is present at about 49.25 mol %, cholesterol is present at about 29.25 mol %, DSPC is present at about 20 mol % and DPG-PEG2K is present at about 1.5 mol %.

In some aspects, the lipid nanoparticle has a mean diameter of about 60 nm to about 100 nm.

In some aspects, the lipid nanoparticle has a mean diameter of about 80 nm.

In some aspects, the lipid nanoparticle has a polydispersity index (PDI) of about 0.05 to about 1.

In some aspects, the lipid nanoparticle has a PDI of about 0.09.

In some aspects, the lipid nanoparticle has a zeta potential (ZP) of about-30 mV to about +5 mV.

In some aspects, the lipid nanoparticle has a ZP of about −1.4 mV.

In some aspects, the cargo comprises one or more nucleic acids.

In some aspects, the one or more nucleic acids is a DNA.

In some aspects, the one or more nucleic acid is an RNA.

In some aspects, the RNA comprises a guide RNA and an RNA that encodes a Cas enzyme.

In some aspects, the one or more nucleic acid encodes a chimeric antigen receptor (CAR).

In some aspects, the cell-targeting group is coupled to a lipid of the lipid nanoparticle to form a lipid-cell targeting group conjugate.

In some aspects, the cell-targeting group is coupled to the PEG-lipid.

In some aspects, the cell-targeting group is coupled to a DPG-PEG2K, a DSPE-PEG2K or a DSPE-PEG3.4K.

In some aspects, the cell-targeting group comprises an antibody or fragment thereof.

In some aspects, the cell-targeting group comprises an antibody.

In some aspects, the cell-targeting group comprises a Fab fragment.

In some aspects, the cell-targeting group comprises a single variable domain.

In some aspects, the cell-targeting group binds a molecule on an immune cell.

In some aspects, the molecule on the immune cell is selected from the group consisting of CD3, CD4, CD7, and CD8.

In some aspects, the cell-targeting group binds a molecule on a hematopoietic stem cell.

In some aspects, the molecule on the hematopoietic stem cell is selected from the group consisting of CD34, CD105, and CD117.

In some aspects, the lipid-cell targeting group conjugate is present in the lipid nanoparticle in a range of about 0.001 mol % to about 0.5 mol %.

In some aspects, provided is a composition comprising the lipid nanoparticle described herein, and one or more carrier or excipient.

In some aspects, the composition is a pharmaceutical composition.

In some aspects, the carrier or excipient is a pharmaceutically acceptable carrier or excipient.

In some aspects, provided is a method of delivering a nucleic acid to a cell in a subject, the method comprising administering to the subject a lipid nanoparticle described herein, or a composition described herein.

In some aspects, provided is a method of delivering a nucleic acid to an immune cell in a subject, comprising administering to the subject a lipid nanoparticle described herein, or a composition described herein.

In some aspects, provided is a method of delivering a nucleic acid to a hematopoietic stem cell in a subject, comprising administering to the subject a lipid nanoparticle described herein, or a composition described herein.

In some aspects, the polydispersity index composition is a pharmaceutical composition.

In some aspects, provided is a use of a lipid nanoparticle described herein, or a composition described herein in the manufacture of a medicament for delivering a nucleic acid to a target cell.

In some aspects, provided is a use of a lipid nanoparticle described herein, or a composition described herein in the manufacture of a medicament for delivering a nucleic acid to an immune cell.

In some aspects, provided is a use of a lipid nanoparticle described herein, or a composition described herein in the manufacture of a medicament for delivering a nucleic acid to a hematopoietic stem cell.

In some aspects, the composition used is a pharmaceutical composition.

In some aspects, provided is a lipid nanoparticle described herein, or a composition described herein for use in delivering a nucleic acid to a target cell.

In some aspects, provided is a lipid nanoparticle described herein, or a composition described herein for use in delivering a nucleic acid to an immune cell.

In some aspects, provided is lipid nanoparticle described herein, or a composition described herein for use in delivering a nucleic acid to a hematopoietic stem cell.

In some aspects, the composition for use in delivering a nucleic acid to a target cell is a pharmaceutical composition.

In some aspects, provided is a method of treating a disease in a subject, the method comprising administering to a cell of the subject in need thereof a lipid nanoparticle described herein, or a composition described herein, or a lipid nanoparticle for use in delivering a nucleic acid to a cell of the subject or a composition for use in delivering a nucleic acid to a cell of the subject.

In some aspects, provided is a method of treating a disease in a subject, the method comprising administering to an immune cell of the subject in need thereof a lipid nanoparticle described herein, or a composition described herein.

In some aspects, the administering is to a plurality of immune cells.

In some aspects, the protein encoded by the one or more nucleic acids disposed in the lipid nanoparticle is detected in at least more than 60% (or even 80%) of immune cells at 72 hours after the administration of the lipid nanoparticle.

In some aspects, the protein is detected in more immune cells in the subject at 72 hours after administration of the lipid nanoparticle, compared to another subject who received a lipid nanoparticle comprising the helper lipid that is present at 10 mol %.

In some aspects, the protein encoded by the one or more nucleic acids disposed in the lipid nanoparticle increases an effector function of the immune cell or plurality of immune cells compared to an immune cell or plurality of immune cells that are not administered the lipid nanoparticle.

In some aspects, provided is a method of treating a disease in a subject, the method comprising administering to a hematopoietic stem cell of the subject in need thereof a lipid nanoparticle described herein, or a composition described herein.

In some aspects, the lipid nanoparticle is administered to a plurality of hematopoietic stem cells of the subject.

In some aspects, a protein encoded by the one or more nucleic acids disposed in the lipid nanoparticle is detected in at least 80% of hematopoietic stem cells of the plurality of hematopoietic stem cells of the subject.

In some aspects, the protein is detected in about 20% of hematopoietic stem cells administered a lipid nanoparticle comprising the helper lipid that is present at 10 mol %.

In some aspects, the administered composition is a pharmaceutical composition.

In some aspects, provided is a lipid nanoparticle described herein, or a composition described herein, for use in the treatment of a disease in a subject in need thereof.

In some aspects, provided is a lipid nanoparticle described herein, or a composition described herein for use in the treatment of an immune cell-related disease in a subject in need thereof.

In some aspects, provided is a lipid nanoparticle described herein, or a composition described herein for use in the treatment of a hematopoietic stem cell-related disease in a subject in need thereof.

In some aspects, the hematopoietic stem-cell related disease is sickle cell disease.

In some aspects, the composition for use in the treatment of a disease, an immune cell-related disease, or a hematopoietic stem cell-related disease in a subject is a pharmaceutical composition.

### BRIEF DESCRIPTION OF DRAWINGS

FIGS. 1A and 1B show the persistent high efficiency transfection of T cells with lipid nanoparticles containing different amounts of DSPC over 72 hours. FIG. 1A shows the percentage of CAR positive T cells following transfection with lipid nanoparticles containing different amounts of DSPC. FIG. 1B shows the CAR mean fluorescence intensity (MFI) of CD8 positive T cells following transfection with lipid nanoparticles containing different amounts of DSPC.

FIGS. 2A-2C show the killing efficiency of T cells transfected with LNPs containing different amounts of DSPC. FIG. 2A shows the percentage of dead Nam6 target cells following co-culture with T cells transfected with LNPs containing different amounts of DSPC. FIG. 2B shows the percentage of CAR positive cells in the T cells transfected with LNPs containing different amounts of DSPC and used in the killing assay of FIG. 2A. FIG. 2C shows the CAR mean fluorescence intensity (MFI) of CD8 positive cells in the T cells transfected with LNPs containing different amounts of DSPC and used in the killing assay of FIG. 2A.

FIGS. 3A-3C show removal of Nalm6 target cells over 16 days in co-cultures with CD8+ T cells transfected with LNPs containing different amounts of DSPC. FIG. 3A shows the mean fluorescence of Nalm6 target cells expressing a red fluorescence protein over a period of 16 days in the presence of CD8+ T cells transfected with LNPs containing different amounts of DSPC. Red arrows indicate additions of Nalm6 target cells to the co-culture; the orange arrow indicates the addition of LNPs to the co-culture. FIG. 3B shows the total count of Nalm6 target cells in the co-cultures at day 16. FIG. 3C shows the percentage of live CD8+ T cells in lymphocytes of the co-cultures at day 16.

FIGS. 4A-4F show the transfection efficiency of lipid nanoparticles containing different amounts of DSPC in T cells of different donors. FIG. 4A shows the percentage of CAR positive of CD8+ T cells of one donor following transfection with lipid nanoparticles containing different amounts of DSPC. FIG. 4B shows the CAR mean fluorescence intensity (MFI) of CD8 positive T cells of the same donor following transfection with lipid nanoparticles containing different amount of DSPC. FIG. 4C shows the percentage of CAR positive of CD8+ T cells of a second donor following transfection with lipid nanoparticles containing different amounts of DSPC. FIG. 4D shows the CAR mean fluorescence intensity (MFI) of CD8 positive T cells of the second donor following transfection with lipid nanoparticles containing different amounts of DSPC. FIG. 4E shows the CAR mean fluorescence intensity (MFI) of CAR positive T cells of the first donor following transfection with lipid nanoparticles containing different amounts of DSPC. FIG. 4F shows the CAR mean fluorescence intensity (MFI) of CAR positive T cells of the second donor following transfection with lipid nanoparticles containing different amounts of DSPC. Luc—Luciferase Reporter as the negative control.

FIGS. 5A-5C show the fluorescence intensity of CAR positive T cells transfected with lipid nanoparticles containing different amounts of DSPC and bound to immobilized CAR targets. FIG. 5A shows the MFI results 24 hours after T cell transfection with LNPs containing different amounts of DSPC. FIG. 5B shows the MFI results 48 hours after T cell transfection with LNPs containing different amounts of DSPC. FIG. 5C shows the MFI results 72 hours after T cell transfection with LNPs containing different amounts of DSPC. In FIGS. 5A-5C: the MFI histograms represent, from left to right, untreated, 10% DSPC, 12.5% DSPC, 15% DSPC, and 20% DSPC.

FIGS. 6A and 6B show the transfection efficiency in human hematopoietic stem cells (HSCs) in vivo of lipid nanoparticles containing different amounts of DSPC and coated with an HSC-specific Fab. FIG. 6A shows the percentage of mCherry positive HSCs in the bone marrow of human HSC engrafted NSG™ mice treated with lipid nanoparticles encapsulating mCherry mRNA and coated with an HSC-specific Fab. FIG. 6B shows off-target signals in liver, spleen, and lung measured by ELISA and in ovary tissue measured by immune histochemistry (IHC) in human HSC engrafted NSG™ mice treated with lipid nanoparticles encapsulating mCherry mRNA and coated with an HSC-specific Fab.

FIGS. 7A and 7B show in vitro transfection efficiency in hematopoietic stem cells. FIG. 7A shows in vitro transfection efficiency (represented by % mCherry positive HSCs) using LNP Formulation 1 (10% DSPC) and LNP Formulation 2 (20% DSPC) at different concentrations (e.g., from about 0.01 micrograms/mL to about 1 micrograms/mL). FIG. 7B shows fluorescence intensity (MFI) following transfection with Formulation 1 and Formulation 2 containing different amounts of DSPC.

### DETAILED DESCRIPTION

The disclosure provides lipid nanoparticles and lipid nanoparticle compositions comprising an ionizable lipid, a structural lipid, a PEG lipid, a specified amount of a helper lipid, and optionally a cargo for highly efficient delivery of the cargo to target cells. In some aspects, the lipid nanoparticles further comprise a cell targeting group, which optionally is coupled to a lipid of the lipid nanoparticle to form a lipid-cell targeting group conjugate. Also provided are methods of making the lipid nanoparticles and compositions and methods of using the same to deliver cargo to specified target cells including immune cells and hematopoietic stem cells.

### Definitions

The term “and/or,” as used herein is a specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). The use of the term “or” means “and/or” unless explicitly indicated to refer to alternatives only, or the alternatives are mutually exclusive.

The expression “at least one of,” as used herein, includes individually each of the recited objects after the expression and the various combinations of two or more of the recited objects unless otherwise understood from the context and use.

The term “approximately” or “about,” as applied to one or more values of interest, refers to a value that is similar to a stated reference value and within a range of values that fall within 10% or less in either direction (10% greater than or 10% less than) of the stated reference value unless otherwise stated or otherwise evident from the context (except where such number would exceed 100% of a possible value). When the term “approximately” or “about” is applied herein to a particular value, the value without the term “approximately” or “about” is also disclosed herein. Further, although not always explicitly stated, all numerical designations may be preceded by the term “about.”

As described herein, any concentration range, percentage range, ratio range, or integer range includes the value of any integer within the recited range and, when appropriate, fractions thereof (such as one tenth and one hundredth of an integer), unless otherwise indicated. The description of an interval of values should be considered as specifically disclosing all possible intermediate intervals as well as each of the values within this interval. For example, the description of an interval from 1 to 6 should be considered as specifically describing each of the intervals that it comprises, such as the intervals from 1 to 3, from 1 to 4, from 1 to 5, from 2 to 4, from 2 to 6, from 3 to 6, etc., as well as each of the values in this interval, for example 1, 2, 2.7, 3, 4, 5, 5.3 and 6. This definition is valid independently of the scope of the interval.

The terms “include,” “includes,” “including,” “have,” “has,” “having,” “contain,” “contains,” or “containing,” “comprise,” “comprises,” or “comprising,” including grammatical equivalents thereof, as used herein, should be understood generally as open-ended and non-limiting, for example, not excluding additional unrecited elements or steps, unless otherwise specifically stated or understood from the context.

The term “lipid nanoparticle” or “LNP,” as used herein, refers to a nanoparticle comprising a single layer of one or more types of lipids. In some aspects, the lipid nanoparticle has a mean diameter of about 400 nm to about 50 nm; or about 400 nm or less, about 350 nm or less, about 300 nm or less, about 250 nm or less, about 200 nm or less, about 150 nm or less, about 100 nm or less, or about 50 nm or less. In some aspects, the nanoparticle has a diameter of about 200 nm to about 50 nm. In some aspects, the nanoparticle has a diameter of about 80 nm.

The term “total lipids,” as used herein, refers to the collection of ionizable lipids, structural lipids, helper lipids, and PEG lipids present in a lipid nanoparticle described herein.

The term “ionizable lipid,” as used herein, refers to a lipid that is capable of modulating its charge depending on the environment it is present in. In some aspects, an ionizable lipid includes a lipid of Formula I, Formula II (Lipid 15), Formula III (KC2), Formula IV (KC3), Formula (V), or Formula (VI). Other ionizable lipids can be used in the lipid nanoparticles described herein.

The terms “helper lipid,” “neutral lipid,” “phospholipid,” or “neutral phospholipid,” as used herein, refer to a non-polar lipid of a lipid nanoparticle which lipid provides the lipid nanoparticle with stability, blood compatibility, and enhances cargo delivery.

The term “PEG lipid” or “PEGylated lipid” are used herein interchangeably and refer to one or more lipids that are modified with polyethylene glycol. As described herein, PEG lipids can be free PEG lipids or PEG lipids that are part of a lipid-cell targeting conjugate.

The term “lipid-cell targeting group conjugate,” as used herein, refers to a conjugate of the general formula: [Lipid]-[optional linker]-[cell targeting group], wherein the lipid can be any lipid described herein and the cell targeting group can be any cell surface molecule-binding group described herein. A cell targeting group includes an antibody or fragment thereof, including a Fab and a scFv, as well as an immunoglobulin single variable domain (such as but not limited to a VHH domain, humanized VHH domain, or camelized VH domain). A lipid of a lipid-cell targeting group conjugate preferably is a PEG lipid.

The term “free PEG lipid,” as used herein, refers to a PEG lipid that is not part of a lipid-cell targeting group conjugate. For example, a free PEG lipid can be part of a lipid blend of a lipid nanoparticle. A free PEG lipid can reduce or eliminate non-specific binding via a targeting group when, e.g., a lipid-immune cell targeting group conjugate is included in a lipid nanoparticle.

The term “linker” or “spacer,” as used herein, denotes a peptide that fuses together two or more polypeptides or proteins into a single molecule. The use of linkers to connect two or more (poly) peptides is well known in the art. For example, a class of peptidic linkers are known as the “Gly-Ser” or “GS” linkers and essentially consist of glycine (G) and serine (S) residues. A linker can also be an alanine linker.

The term “coupled,” as used herein refers to a covalent interaction between two molecules. Therefore, “coupled” includes “covalently coupled.”

The term “antibody,” as used herein, refers to any antigen-binding molecule or molecular complex comprising at least one complementarity determining region (CDR) that specifically binds to or interacts with a particular antigen. It is understood that the term encompasses an intact antibody, antigen-binding fragment thereof, or an Fc fragment that optionally has been modified or engineered. Examples of antigen binding fragments include Fab, Fab′, (Fab′)2, Fv, single chain antibodies (e.g., scFv), minibodies, immunoglobulin single variable domains (e.g., VHHs), and diabodies. Examples of antibodies that have been modified or engineered include chimeric antibodies, humanized antibodies, and multi-specific antibodies (e.g., bispecific antibodies).

Naturally occurring antibodies typically comprise a tetramer. Each such tetramer is typically composed of two identical pairs of polypeptide chains, each pair having one full length light chain (typically having a molecular weight of about 25 kDa) and one full length heavy chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (VH) and three constant domains (CH1, CH2, and CH3), wherein the VH domain is at the amino-terminus of the polypeptide and the CH3 domain is at the carboxyl-terminus, and a full-length light chain immunoglobulin polypeptide includes a variable domain (VL) and a constant domain (CL), wherein the VL domain is at the amino-terminus of the polypeptide and the CL domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety herein. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol. Biol. 196:901-17; Chothia et al., 1989, Nature 342:877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9:133-39; MacCallum, 1996, J. Mol. Biol. 262(5): 732-45; and Lefranc, 2003, Dev. Comp. Immunol. 27:55-77. Still other CDR boundary definitions may not strictly follow one of the mentioned systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain aspects use Kabat or Chothia defined CDRs. Identification of predicted CDRs using the amino acid sequence is well known in the field, such as in Martin, A. C. Protein sequence and structure analysis of antibody variable domains, In Antibody Engineering, Vol. 2. Kontermann R., Dübel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments.

The term “Fc,” as used herein, refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins, although IgG1 and IgG2 are preferred. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a Fc is a disulfide-bonded dimer resulting from papain digestion of an IgG. The term “native Fc” as used herein is generic to the monomeric, dimeric, and multimeric forms.

The term “Fab,” as used herein, refers to a F (ab) fragment of an antibody and typically includes one light chain and the VH and CH1 domains of one heavy chain, wherein the VH-CH1 heavy chain portion of the F (ab) fragment cannot form a disulfide bond with another heavy chain polypeptide. As used herein, a F (ab) fragment can also include one light chain containing two variable domains separated by an amino acid linker and one heavy chain containing two variable domains separated by an amino acid linker and a CH1 domain. A F (ab′) fragment typically includes one light chain and a portion of one heavy chain that contains more of the constant region (between the CH1 and CH2 domains), such that an interchain disulfide bond can be formed between two heavy chains to form a F(ab′)2 molecule.

The term “immunoglobulin single variable domain,” as used herein, refers to an immunoglobulin domain capable of specifically binding to an epitope of the antigen without pairing with an additional immunoglobulin variable domain. The binding site of an immunoglobulin single variable domain is formed by a single VH, a single VHH or single VL domain. Therefore, the antigen binding site of an immunoglobulin single variable domain is formed by no more than three CDRs. The single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). For example, an immunoglobulin single variable domain can be a heavy chain immunoglobulin single variable domain, such as a VH, VHH, including a camelized VH or humanized VHH or a “dAb” or dAb (or an amino acid sequence that is suitable for use as a dAb). In some aspects, an immunoglobulin single variable domain is a VHH, including a camelized VH or humanized VHH. In some aspects, single variable domains are derived from certain species of shark (for example, the so-called “IgNAR domains.” In some aspects, an immunoglobulin single variable domain is a NANOBODY® protein. [Note: NANOBODY® is a registered trademark of Ablynx N.V.]

The term “scFv,” as used herein, refers to a single chain antibody comprising a heavy chain variable domain and a light chain variable domain linked by a linker.

The term “VHH” is used herein in its broadest sense and is not limited to a specific biological source or to a specific method of preparation. For example, a VHH can be obtained (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, in particular a species of mammal, such as from a human, or by expression of a nucleic acid encoding such a camelized VH domain; (5) by “camelization” of a “domain antibody” or “dAb” or by expression of a nucleic acid encoding such a camelized VH domain; (6) using synthetic or semi-synthetic techniques for preparing proteins, polypeptides or other amino acid sequences; (7) by preparing a nucleic acid encoding a VHH using techniques for nucleic acid synthesis, followed by expression of the nucleic acid thus obtained; and/or (8) by any combination of the foregoing.

In some aspects, immunoglobulin sequences of different origin, comprising mouse, rat, rabbit, donkey, human and camelid immunoglobulin sequences are used in the cell targeting groups described herein. Also, fully human, humanized or chimeric sequences can be used in the cell targeting groups described herein.

The term “humanized antibody,” as used herein, refers to an antibody which is wholly or partially of non-human origin and whose protein sequence has been modified to replace certain amino acids, for instance that occur at the corresponding position(s) in the framework regions of the VH and VL domains in a sequence of an antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. In some aspects, the constant domains of a humanized antibody are human CH and CL domains.

The term “humanized VHH,” as used herein, refers to an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional four-chain antibody from a human.

The term “camelized VH,” as used herein, refers to an amino acid sequence that corresponds to the amino acid sequence of a naturally occurring VH domain, but that has been “camelized,” i.e. by replacing one or more amino acid residues in the amino acid sequence of a naturally occurring VH domain from a conventional four-chain antibody by one or more of the amino acid residues that occur at the corresponding position(s) in a VHH domain of a (camelid) heavy chain antibody. In some aspects, such “camelizing” substitutions are inserted at amino acid positions that form and/or are present at the VH-VL interface. In some aspects, the VH sequence that is used as a starting material or starting point for generating or designing the camelized VH is a VH sequence from a mammal, such as the VH sequence of a human, such as, e.g., a VH3 sequence. However, it should be noted that such camelized VH can be obtained in any suitable manner and is not strictly limited to a polypeptide that has been obtained using a polypeptide that comprises a naturally occurring VH domain as a starting material. For example, camelid immunoglobulin sequences and humanized camelid immunoglobulin sequences, or camelized domain antibodies, e.g., camelized dAb can be used herein. In some aspects, immunoglobulin single variable domains are fused forming a multivalent and/or multispecific construct (for multivalent and multispecific polypeptides containing one or more VHH domains).

The term “multivalent,” as used herein, refers to the presence of multiple immunoglobulin single variable domains in a polypeptide. In some aspects, the polypeptide is “bivalent,” i.e., comprises or consists of two immunoglobulin single variable domains. In some aspects, the polypeptide is “trivalent,” i.e., comprises or consists of three immunoglobulin single variable domains. In some aspects, the polypeptide is “tetravalent,” i.e. comprises or consists of four immunoglobulin single variable domains. The polypeptide can thus be “bivalent,” “trivalent,” “tetravalent,” “pentavalent,” “hexavalent,” “heptavalent,” “octavalent,” “nonavalent,” etc., i.e., the polypeptide comprises or consists of two, three, four, five, six, seven, eight, nine, etc., immunoglobulin single variable domains, respectively. In some aspects, a multivalent immunoglobulin single variable domain polypeptide is trivalent. In some aspects, a multivalent immunoglobulin single variable domain polypeptide is tetravalent. In some aspects, the multivalent immunoglobulin single variable domain polypeptide is pentavalent. In some aspects, a multivalent immunoglobulin single variable domain polypeptide can also be multispecific.

The term “multispecific,” as used herein, refers to binding to multiple different target molecules (also referred to as antigens). In some aspects, a multivalent immunoglobulin single variable domain polypeptide is “bispecific,” “trispecific,” “tetraspecific,” etc., i.e., can bind to two, three, four, etc., different target molecules, respectively. For example, a polypeptide may be bispecific-trivalent, such as a polypeptide comprises or consists of three immunoglobulin single variable domains, wherein two immunoglobulin single variable domains bind to a first target and one immunoglobulin single variable domain binds to a second target different from the first target. In some aspects, a polypeptide is trispecific-tetravalent, such as a polypeptide comprises or consists of four immunoglobulin single variable domains, wherein, e.g., one immunoglobulin single variable domain binds to a first target, two immunoglobulin single variable domains bind to a second target different from the first target and one immunoglobulin single variable domain binds to a third target different from the first and the second target. In some aspects, a polypeptide is trispecific-pentavalent, such as a polypeptide comprises or consists of five immunoglobulin single variable domains, wherein, e.g., two immunoglobulin single variable domains bind to a first target, two immunoglobulin single variable domains bind to a second target different from the first target and one immunoglobulin single variable domain binds to a third target different from the first and the second target. In some aspects, a multispecific immunoglobulin polypeptide is multiparatopic.

The term “multiparatopic,” as used herein, refers to binding to multiple different epitopes on the same target molecules (also referred to as antigens). In some aspects, a multivalent immunoglobulin single variable domain polypeptide can thus be “biparatopic,” “triparatopic,” etc., i.e., can bind to two, three, etc., different epitopes on the same target molecules, respectively.

The terms “subject” and “patient,” as used herein, refer to organisms that are preferably mammals (e.g., murines, simians, equines, bovines, porcines, canines, felines, and the like), and more preferably humans.

The term “pharmaceutical composition,” as used herein, refers to the combination of an active agent with a carrier, inert or active, making the composition especially suitable for diagnostic or therapeutic use in vivo or ex vivo.

The term “pharmaceutically acceptable carrier or excipient,” as used herein, refers to any of the standard pharmaceutical excipient, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, MD, 2006.

The term “effective amount,” as used herein, refers to the amount of a compound (e.g., a nucleic acid) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount,” as used herein, refers to an amount of a lipid nanoparticle comprising, e.g., a nucleic acid or a composition comprising a lipid nanoparticle, which is effective for producing a desired therapeutic effect in at least a sub-population of cells in a mammal, for example, a human, or a subject (e.g., a human subject) at a reasonable benefit/risk ratio applicable to any medical treatment.

The phrase “prophylactically effective amount,” as used herein, refers to an amount of a lipid nanoparticle comprising, e.g., a nucleic acid or composition comprising a lipid nanoparticle, which is effective for producing a desired prophylactic effect in at least a sub-population of cells in a mammal, for example, a human, or a subject (e.g., a human subject) by reducing, minimizing or eliminating the risk of developing a condition or by reducing or minimizing severity of a condition at a reasonable benefit/risk ratio applicable to any medical treatment.

The terms “treat,” “treating,” and “treatment” include any effect, e.g., lessening, reducing, modulating, ameliorating or eliminating, that results in the improvement of a condition, disease, disorder, and the like, or ameliorating a symptom thereof.

### Lipids

Provided herein are lipid nanoparticles comprising a lipid blend comprising one or more of the following lipids: ionizable lipid, structural lipid, helper lipid, and PEG lipid.

In some aspects, an ionizable lipid comprises a structure of Formula I:

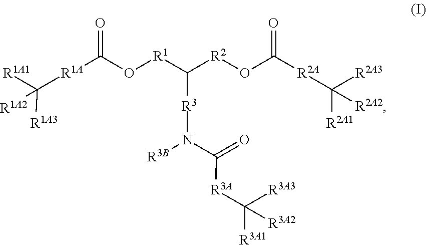

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀₀C(O)R^{a2}; R^{a1} and R^{a2} are each independently C₁₋₂₀ alkyl or C₁₋₂₀ alkenyl; R^{3B} is

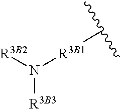

R^{3B1} is C₁₋₆ alkylene; and
R^{3B2} and R^{3B3} are each independently H or C₁₋₆ alkyl.

In some aspects, an ionizable lipid has been designed to enable intracellular delivery of a cargo, e.g., a nucleic acid to the cytosolic compartment of a target cell type and rapidly degrade into non-toxic components. The complex functionalities of the ionizable cationic lipid are facilitated by the interplay between the chemistry and geometry of the ionizable lipid head group, the hydrophobic “acyl-tail” groups and the linkers connecting the head group and the acyl tail groups.

In some aspects, the pKa of the ionizable amine head group is designed to be in the range of 6-8, such as between 6.2-7.4, or between 6.7-7.2, such that it remains strongly cationic under acidic formulation conditions (e.g., pH 4-pH 5.5), neutral or slightly anionic in physiological pH (7.4) and cationic in the early and late endosomal compartments (e.g., pH 5.5-pH 7).

In some aspects, the acyl-tail groups of an ionizable lipid are designed to enable fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid cargo). In some aspects, an ionizable lipid comprises a linker connecting the head group and acyl tail groups which linker is designed to degrade by physiologically prevalent enzymes (e.g., esterases, or proteases) or by acid catalyzed hydrolysis.

In some aspects, an ionizable lipid comprises a structure of Formula II (Lipid 15):

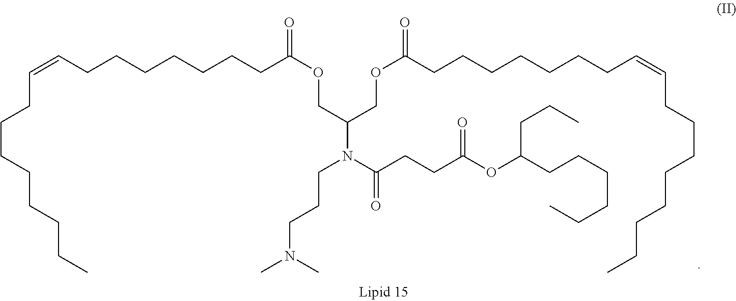 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In some aspects, an ionizable lipid comprises a structure of Formula III (KC2).

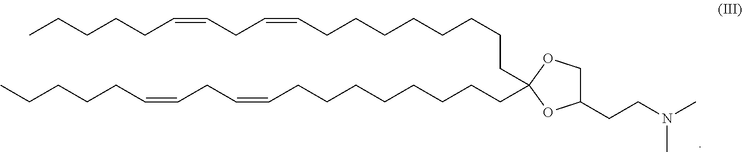 [SMILES: CCCCC/C=C\C/C=C\CCCCCCCCC1(CCCCCCCC/C=C\C/C=C\CCCCC)OCC(CCN(C)C)O1]

In some aspects, an ionizable lipid comprises a structure of Formula IV (KC3):

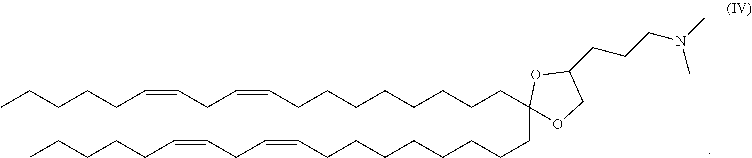 [SMILES: CCCCC/C=C\C/C=C\CCCCCCCCC1(CCCCCCCC/C=C\C/C=C\CCCCC)OCC(CCCN(C)C)O1]

In some aspects, the ionizable lipid comprises a structure of Formula V:

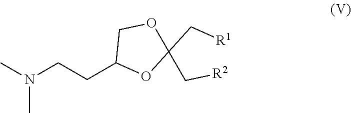

wherein R¹ and R² are each independently a lipid.

In some aspects, the ionizable lipid comprises a structure of Formula VI:

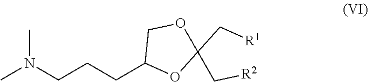

wherein R¹ and R² are each independently a lipid.

In some embodiments, where the ionizable lipid comprises a structure of Formula V or Formula VI, R¹ and R² are each independently substituted or unsubstituted alkyl, substituted or unsubstituted alkenyl. In some embodiments, where the ionizable lipid comprises a structure of Formula V or Formula VI, R¹ and R² are the same lipid. In some embodiments, where the ionizable lipid comprises a structure of Formula V or Formula VI, R¹ and R² are different lipids. In some embodiments, the lipid is a hydrocarbon (e.g., substituted or unsubstituted, saturated or unsaturated, branched or unbranched hydrocarbon). A hydrocarbon may be an alkane, alkene, or alkyne. In some embodiments, the hydrocarbon chain is saturated or unsaturated. In certain embodiments, an unsaturated hydrocarbon chain comprises at least one, at least one two, at least one three, at least one four, at least one five, or at least one six carbon-carbon double bonds (e.g., cis double bonds and/or trans double bonds). In some embodiments, the lipid is substituted or unsubstituted C₇₋₃₆ alkyl. In certain embodiments, a lipid is substituted or unsubstituted C₇₋₃₆ alkenyl. In certain embodiments, the lipid is unsubstituted C₇₋₃₆ alkyl or unsubstituted C₇₋₃₆ alkenyl. In certain embodiments, a hydrocarbon is substituted with alkyl, alkenyl, alkynyl, amino, alkylamino, dialkylamino, trialkylamino, hydroxyl, alkoxy, carbonyl, halogen, aryl, heterocyclic, aromatic heterocyclic, cyano, amide, carbamoyl, carboxylic acid, ester, thioether, alkylthioether, thiol, ureido, or a combination thereof. Each of these groups may in turn be substituted. In some embodiments, a hydrocarbon is substituted with one, two, three, four, five, six, seven, eight, nine, ten, or more than ten substituents.

In some aspects, the ionizable lipid comprises one or more lipids as described in International PCT Publication Nos. WO2009028824A2, WO2011043913A2, WO2012091523A2, WO2015074085A1, WO2016081029A1, WO2017117530A1, WO2018118102A1, WO2018119163A1, WO2019045897A1, WO2020069445A1, WO2020106903A1, WO2020219876A1, WO2021055892A1, WO2021163339A1, WO2021188389A2, WO2021202694A1, WO2022016070A1, WO2022119883A2, WO2022120388A2, WO2022159475A1, WO2022207938A1, WO2022218957A1, WO2022246555A1, WO2023023410A2, WO2023091490A1, WO2023091787A1, WO2023114937A2, WO2023114944A1, WO2023115221A1, WO2023121970A1, WO2023121975A1, WO2023141624A1, WO2023144798A1, WO2023183616A1, WO2023196444A1, WO2023196527A2, WO2023207101A1, WO2023235589A1, WO2023240156A1, WO2024008967A1, WO2024010330A1, WO2024019770A1, and WO2024035710A2. In other aspects, the ionizable lipid comprises one or more lipids as described in Semple, S., Akinc, A., Chen, J. et al. Rational design of cationic lipids for siRNA delivery. Nat Biotechnol 28, 172-176 (2010).

In some aspects, an ionizable lipid is present in the lipid blend of a lipid nanoparticle in a range of about 20 mol % to about 70 mol %.

In some aspects, an ionizable lipid is present in the lipid blend of a lipid nanoparticle in a range of about 20 mol % to about 50 mol %. In some aspects, an ionizable lipid is present in the lipid blend of a lipid nanoparticle at about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, about 65 mol %, about 66 mol %, about 67 mol %, about 68 mol %, about 69 mol %, or about 70 mol %.

In some aspects, an ionizable lipid is present in the lipid blend of a lipid nanoparticle at range of about 25 mol % to about 65 mol %; about 30 mol % to about 60 mol %; about 35 mol % to about 55 mol %; or about 40 mol % to about 50 mol %. In some aspects, an ionizable lipid is present in the lipid blend of a lipid nanoparticle at range of about 40 mol % to about 60 mol %; about 45 mol % to about 55 mol %; or about 48 mol % to about 50 mol %. In some aspects, the ionizable lipid is present in the lipid blend of a lipid nanoparticle at about 49 mol %. In some aspects, the lipid blend of a lipid nanoparticle comprises at least 20 mol % ionizable lipid. In some aspects, the lipid blend of a lipid nanoparticle comprises at least 30 mol % ionizable lipid. In some aspects, the lipid blend of a lipid nanoparticle comprises at least 40 mol % ionizable lipid. In some aspects, the lipid blend of a lipid nanoparticle comprises at least 50 mol % ionizable lipid.

In some aspects, a lipid nanoparticle comprises one or more helper lipids. In some aspects, a helper lipid is a phospholipid. In some aspects, a helper lipid is a neutral phospholipid. In some aspects, a helper lipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM), dimyrstoyl-phosphatidylethanolamine (DMPE), dilinoleoyl-glycero-phosphocholine (DLPC), dimyristoyl-glycero-phosphocholine (DMPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, or sphingomyelin.

In some aspects, a helper lipid is present in the lipid blend of a lipid nanoparticle in a range of about 10 mol % to about 50 mol %. In some aspects, a helper lipid is present in the lipid blend of a lipid nanoparticle at about 10 mol %, about 11 mol %, about 12 mol %, about 13 mol %, about 14 mol %, or about 15 mol %, at about 16 mol %, about 17 mol %, about 18 mol %, about 19 mol %, about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, or about 50 mol %. In some aspects, a helper lipid is present in the lipid blend of a lipid nanoparticle in a range of about 16 mol % to about 50 mol %. In some aspects, a helper lipid is present in the lipid blend of a lipid nanoparticle at about 16 mol % to about 40 mol %. In some aspects, a helper lipid is present in the lipid blend of a lipid nanoparticle at about 16 mol % to about 35 mol %. In some aspects, a helper lipid is present in the lipid blend of a lipid nanoparticle at about 16 mol % to about 30 mol %. In some aspects, a helper lipid is present in the lipid blend of a lipid nanoparticle at about 16 mol % to about 25 mol %. In some aspects, a helper lipid is present in the lipid blend of a lipid nanoparticle at about 16 mol % to about 20 mol %. In some aspects, a helper lipid is present in the lipid blend of a lipid nanoparticle at about 18 mol % to about 25 mol %. In some aspects, a helper lipid is present in the lipid blend of a lipid nanoparticle at about 18 mol % to about 22 mol %. In some aspects, a helper lipid is present in the lipid blend of a lipid nanoparticle at about 20 mol % to about 40 mol %. In some aspects, a helper lipid is present in the lipid blend of a lipid nanoparticle at about 20 mol % to about 35 mol %. In some aspects, a helper lipid is present in the lipid blend of a lipid nanoparticle at about 20 mol % to about 30 mol %. In some aspects, a helper lipid is present in the lipid blend of a lipid nanoparticle at about 20 mol % to about 25 mol %. In some aspects, a helper lipid is present in the lipid blend of a lipid nanoparticle at about 25 mol % to about 40 mol %. In some aspects, a helper lipid is present in the lipid blend of a lipid nanoparticle at about 25 mol % to about 35 mol %. In some aspects, a helper lipid is present in the lipid blend of a lipid nanoparticle at about 25 mol % to about 30 mol %. In some aspects, a helper lipid is present in the lipid blend of a lipid nanoparticle at about 30 mol % to about 40 mol %. In some aspects, a helper lipid is present in the lipid blend of a lipid nanoparticle at about 30 mol % to about 35 mol %. In some aspects, a helper lipid is present in the lipid blend of a lipid nanoparticle at about 20 mol %. In some aspects, a helper lipid is present in the lipid lend of a lipid nanoparticle at about 30 mol %. In some aspects, a helper lipid is present in the lipid blend of a lipid nanoparticle at about 40 mol %. In some aspects, the lipid blend of a lipid nanoparticle comprises at least 15 mol % helper lipid. In some aspects, the lipid blend of a lipid nanoparticle comprises at least 20 mol % helper lipid. In some aspects, the lipid blend of a lipid nanoparticle comprises at least 30 mol % helper lipid.

In some aspects, a helper lipid of the lipid nanoparticle is DSPC.

In some aspects, a lipid nanoparticle comprises one or more PEG lipids.

In some aspects, a PEG lipid is a PEG-modified phosphatidylethanolamine, a PEG-modified phosphatidic acid, a PEG-modified ceramide, a PEG-modified dialkylamine, a PEG-modified diacylglycerol, or a PEG-modified dialkylglycerol.

In some aspects, a PEG lipid is selected from the group consisting of dioleoylgylcerol-PEG (DOG-PEG), dimyristoyl-glycerol-PEG (DMG-PEG), dipalmitoyl-glycerol-PEG (DPG-PEG), dilinoleoyl-glycero-phosphatidyl ethanolamine-PEG (DLPE-PEG), dimyrstoyl-phosphatidylethanolamine-PEG (DMPE-PEG), dipalmitoyl-phosphatidylethanolamine-PEG (DPPE-PEG), distearoylglycerol-PEG (DSG-PEG), diacylglycerol-PEG (DAG-PEG), dimyristoyl-glycerol-PEG (DMG-PEG), dipalmitoyl-glycerol (DPG-PEG), distearoylglycerol-PEG (DSG-PEG), PEG-ceramide, distearoyl-glycero-phosphoglycerol-PEG (DSPG-PEG), dioleoyl-glycero-phosphoethanolamine-PEG (DOPE-PEG), 2-[(polyethylene glycol)-2000]-N,N-ditetradecylacetamide, and distearoyl-phosphatidylethanolamine-PEG (DSPE-PEG), DSPE-PEG-cysteine, or a derivative thereof. In some aspects, the PEG lipid comprises a diacylphosphatidylethanolamine comprising a Dipalmitoyl (C16) chain or a Distearoyl (C18) chain.

In some aspects, a PEG lipid is present in the lipid blend of a lipid nanoparticle in a range of about 1 mol % to about 10 mol %, about 1 mol % to about 9 mol %, about 1 mol % to about 8 mol %, about 1 mol % to about 7 mol %, about 1 mol % to about 6 mol %, about 1 mol % to about 5 mol %, about 1 mol % to about 4 mol %, about 1 mol % to about 3 mol %. In some aspects, a PEG lipid is present in the lipid blend of a lipid nanoparticle in a range of about 2 mol % to about 10 mol %, about 2 mol % to about 9 mol %, about 2 mol % to about 8 mol %, about 2 mol % to about 7 mol %, about 2 mol % to about 6 mol %, about 2 mol % to about 5 mol %, about 2 mol % to about 4 mol %, about 2 mol % to about 3 mol %, or about 1 mol %, about 2 mol %, about 3 mol % about 4 mol % or about 5 mol %. In some aspects, a PEG lipid is a free PEG lipid.

In some aspects, a PEG lipid is present in the lipid blend of a lipid nanoparticle in a range of about 0.01 mol % to about 10 mol %, about 0.01 mol % to about 5 mol %, about 0.01 mol % to about 4 mol %, about 0.01 mol % to about 3 mol %, about 0.01 mol % to about 2 mol %, about 0.01 mol % to about 1 mol %, about 0.1 mol % to about 10 mol %, about 0.1 mol % to about 5 mol %, about 0.1 mol % to about 4 mol %, about 0.1 mol % to about 3 mol %, about 0.1 mol % to about 2 mol %, about 0.1 mol % to about 1 mol %, about 0.5 mol % to about 10 mol %, about 0.5 mol % to about 5 mol %, about 0.5 mol % to about 4 mol %, about 0.5 mol % to about 3 mol %, about 0.5 mol % to about 2 mol %, about 0.5 mol % to about 1 mol %, about 1 mol % to about 2 mol %, about 3 mol % to about 4 mol %, about 4 mol % to about 5 mol %, about 5 mol % to about 6 mol %, or. In some aspects, a PEG lipid is present in the lipid blend of a lipid nanoparticle at about 0.5 mol %, about 1 mol %, about 1.5 mol %, about 2 mol %, about 2.5 mol %, about 3 mol %, about 3.5 mol %, about 4 mol %, about 4.5 mol %, about 5 mol %, or about 5.5 mol %. In some aspects, the PEG lipid is a free PEG lipid.

In some aspects, a PEG lipid is present in the lipid blend of a lipid nanoparticle in a range of about 0.1 mol % to about 4 mol %. In some aspects, a PEG lipid is present in the lipid blend of a lipid nanoparticle in a range of about 0.5 mol % to about 3.5 mol %, about 0.75 mol % to about 3 mol %, about 1 mol % to about 2.5 mol %, about 1.5 mol % to about 2 mol %. In some aspects, a PEG lipid is present in the lipid blend of a lipid nanoparticle in an amount of about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3 mol %, about 3.1 mol %, about 3.2 mol %, about 3.3 mol %, about 3.4 mol %, about 3.5 mol %, about 3.6 mol %, about 3.7 mol %, about 3.8 mol %, about 3.9 mol %, or about 4 mol %. In some aspects, a PEG lipid is present in the lipid blend of a lipid nanoparticle at about 1.25 mol % to about 1.75 mol %. In some aspects, a PEG lipid is present in the lipid blend of a lipid nanoparticles at about 1.5 mol %.

In some aspects, the lipid blend of a lipid nanoparticle comprises at least 0.5 mol % PEG lipid. In some aspects, the lipid blend of a lipid nanoparticle comprises at least 1 mol % PEG lipid. In some aspects, the lipid blend of a lipid nanoparticle comprises at least 2 mol % PEG lipid.

In some aspects, a lipid nanoparticle comprises one or more structural lipids. In some aspects, a structural lipid is a sterol. In some aspects, one or more sterols are selected from the group consisting of cholesterol, fecosterol, β-sitosterol, ergosterol, campesterol, stigmasterol, stigmastanol, brassicasterol. In some aspects, the sterol is cholesterol.

In some aspects, a structural lipid is present in the lipid blend of a lipid nanoparticle in a range of about 20 mol % to about 70 mol %.

In some aspects, a structural lipid is present in the lipid blend of a lipid nanoparticle in a range of about 20 mol % to about 50 mol %. In some aspects, a structural lipid is present in the lipid blend of a lipid nanoparticle at about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, about 65 mol %, about 66 mol %, about 67 mol %, about 68 mol %, about 69 mol %, or about 70 mol %.

In some aspects, a structural lipid is present in the lipid blend of a lipid nanoparticle at range of about 20 mol % to about 65 mol %; about 20 mol % to about 60 mol %; about 20 mol % to about 55 mol %; or about 25 mol % to about 50 mol %, or about 25 mol % to about 45 mol %, about 20 mol % to about 40 mol %. In some aspects, a structural lipid is present in the lipid blend of a lipid nanoparticle at about 20%, about 30 mol %, about 40 mol %, or about 50 mol %. In some aspects, a structural lipid is present in the lipid blend of a lipid nanoparticle at about 29 mol %. In some aspects, a structural lipid is present in the lipid blend of a lipid nanoparticle at about 39 mol %. In some aspects, the lipid blend of a lipid nanoparticle comprises at least 20 mol % structural lipid. In some aspects, the lipid blend of a lipid nanoparticle comprises at least 25 mol % structural lipid. In some aspects, the lipid blend of a lipid nanoparticle comprises at least 30 mol % structural lipid.

In some aspects, a lipid nanoparticle comprises an ionizable lipid that is present in the lipid blend of the lipid nanoparticle in a range of about 30 mol % to about 70 mol %; a helper lipid that is present in the lipid blend of the lipid nanoparticle in a range of about 10 mol % to about 50 mol %; a structural lipid that is present in the lipid blend of the lipid nanoparticle in a range of about 20 mol % to about 70 mol %; a PEG lipid that is present in the lipid blend of the lipid nanoparticle in a range of about 1 mol % to about 10 mol %, and, optionally, a cargo.

In some aspects, a lipid nanoparticle comprises an ionizable lipid that is present in the lipid blend of the lipid nanoparticle in a range of about 40 mol % to about 60 mol %; a helper lipid that is present in the lipid blend of the lipid nanoparticle in a range of about 16 mol % to about 40 mol %; a structural lipid that is present in the lipid blend of the lipid nanoparticle in a range of about 20 mol % to about 50 mol %; a PEG lipid that is present in the lipid blend of the lipid nanoparticle in a range of about 1 mol % to about 5 mol %, and, optionally, a cargo.

In some aspects, a lipid nanoparticle comprises an ionizable lipid that is present in the lipid blend of the lipid nanoparticle in a range of about 48 mol % to about 50 mol %; a helper lipid that is present in the lipid blend of the lipid nanoparticle in a range of about 16 mol % to about 40 mol %; a structural lipid that is present in the lipid blend of the lipid nanoparticle in a range of about 20 mol % to about 40 mol %; a PEG lipid that is present in the lipid blend of the lipid nanoparticle in a range of about 1 mol % to about 2 mol %, and, optionally, a cargo.

In some aspects, a lipid nanoparticle comprises an ionizable lipid that is selected from the group consisting of Formula II (Lipid 15), Formula III (KC2), Formula IV (KC3), Formula V, and Formula VI, and present in the lipid blend of the lipid nanoparticle in a range of about 30 mol % to about 70 mol %; a helper lipid that is DSPC and is present in the lipid blend of the lipid nanoparticle in a range of about 10 mol % to about 50 mol %; a structural lipid that is cholesterol and is present in the lipid blend of the lipid nanoparticle in a range of about 20 mol % to about 70 mol %; a PEG lipid that is DPG-PEG2K and is present in the lipid blend of the lipid nanoparticle in a range of about 1 mol % to about 10 mol %, and, optionally, a cargo.

In some aspects, a lipid nanoparticle comprises an ionizable lipid that is selected from the group consisting of Formula II (Lipid 15), Formula III (KC2), Formula IV (KC3), Formula V, and Formula VI, and is present in the lipid blend of the lipid nanoparticle in a range of about 40 mol % to about 60 mol %; a helper lipid that is DSPC and is present in the lipid blend of the lipid nanoparticle in a range of about 16 mol % to about 40 mol %; a structural lipid that is cholesterol and is present in the lipid blend of the lipid nanoparticle in a range of about 20 mol % to about 50 mol %; a PEG lipid that is DPG-PEG2K and is present in the 1 lipid blend of the lipid nanoparticle in a range of about 1 mol % to about 5 mol %, and, optionally, a cargo.

In some aspects, a lipid nanoparticle comprises an ionizable lipid that is selected from the group consisting of Formula II (Lipid 15), Formula III (KC2), Formula IV (KC3), Formula V, and Formula VI and is present in the lipid blend of the lipid nanoparticle in a range of about 48 mol % to about 50 mol %; a helper lipid that is DSPC and is present in the lipid blend of the lipid nanoparticle in a range of about 16 mol % to about 20 mol %; a structural lipid that is cholesterol and is present in the lipid blend of the lipid nanoparticle in a range of about 20 mol % to about 40 mol %; a PEG lipid that is DPG-PEG2K and is present in the lipid blend of the lipid nanoparticle in a range of about 1 mol % to about 2 mol %, and, optionally, a cargo.

In some aspects, a lipid nanoparticle comprises an ionizable lipid that is selected from the group consisting of Formula II (Lipid 15), Formula III (KC2), Formula IV (KC3), Formula V, and Formula VI and is present in the lipid blend of the lipid nanoparticle in an amount of about 49.24 mol %; a helper lipid that is DSPC and is present in the lipid blend of the lipid nanoparticle in an amount of about 20 mol %; a structural lipid that is cholesterol and is present in the lipid blend of the lipid nanoparticle in an amount of about 29.25 mol %; a PEG lipid that is DPG-PEG2K and is present in the lipid blend of the lipid nanoparticle in an amount of about 1.51 mol %; and, optionally, a cargo.

In some aspects, wherein a lipid nanoparticle includes a lipid blend, the value of mol % refers to the mol % within the lipid blend. For example, wherein a nanoparticle includes a lipid blend that includes an ionizable lipid, a structural lipid, a helper lipid, and a PEG lipid, then the sum of the mol % ionizable lipid, mol % structural lipid, mol % helper lipid, and mol % PEG lipid is 100%.

### Cell Targeting Groups

In some aspects, a lipid nanoparticle described herein comprises a cell targeting group conjugated to a lipid of the lipid nanoparticle. In some aspects, a lipid nanoparticle comprises more than one cell targeting group conjugated to a lipid of the lipid nanoparticle. In some aspects, a cell targeting group comprises an antibody. In some aspects, the antibody is a human or humanized antibody. In some aspects, a cell targeting group comprises an antibody fragment without an Fc component. In some aspects, a cell targeting group comprises an antigen-binding fragment selected from the group consisting of a Fab, F(ab′)2, Fab′-SH, Fv, scFv fragment, or immunoglobin single variable domain. In some aspects, a cell targeting group comprises a Fab. In some aspects, a cell targeting group comprises an immunoglobin single variable domain.

In some aspects, a cell targeting group comprises one or more immunoglobulin single variable domains. In some aspects, a cell targeting group comprises one or more antibody fragments such as Fab, scFv, or combinations thereof.

In some aspects, a cell targeting group is a monovalent, multivalent, or multispecific polypeptide. In some aspects, a cell targeting group comprises a multivalent and multispecific polypeptide that contains one or more antibodies, one or more immunoglobulin single variable domains, such as one or more VHH domain, and/or one or more Fab, and/or one or more scFv.

In some aspects, a multivalent cell targeting group comprises two or more immunoglobulin single variable domains, Fab, or scFv that target the same antigen, e.g., the same part or epitope of said antigen or two or more different parts or epitopes of said antigen. In some aspects, a multivalent cell targeting group comprises two or more immunoglobulin single variable domains, Fab, or scFv that are directed to different antigens. In some aspects, a multivalent cell targeting group comprises a combination of immunoglobulin single variable domains, Fab, or scFv some of which target the same antigen and some of which target different antigens.

In some aspects, a bivalent cell targeting group comprises a first immunoglobulin single variable domain, Fab, or scFv targeting a first part or epitope of an antigen and a second immunoglobulin single variable domain, Fab, or scFv targeting the same part or epitope of said antigen or another part or epitope of said antigen. In some aspects, a bivalent cell targeting group comprises a first immunoglobulin single variable domain, Fab, or scFv targeting a first antigen and a second immunoglobulin single variable domain, Fab, or scFv targeting a second antigen different from said first antigen.

In some aspects, a trivalent cell targeting group comprises three identical or different immunoglobulin single variable domains, Fab, or scFv targeting the same or different parts or epitopes of the same antigen. In some aspects, a trivalent cell targeting group comprises two identical or different immunoglobulin single variable domains, Fab, or scFv targeting the same or different parts or epitopes on a first antigen and a third immunoglobulin single variable domain targeting a second antigen different from said first antigen.

In some aspects, a trivalent cell targeting group comprises a first immunoglobulin single variable domain, Fab, or scFv targeting a first antigen, a second immunoglobulin single variable domain, Fab, or scFv targeting a second antigen different from said first antigen, and a third immunoglobulin single variable domain, Fab, or scFv targeting a third antigen different from said first and second antigen.

In some aspects, a cell targeting group comprises one or more immunoglobulin single variable domains, Fab, or scFv and optionally further comprises one or more further amino acid sequences (all optionally linked via one or more suitable linkers).

In some aspects, a cell targeting group further comprises one or more other groups, residues, moieties or binding units. Such further groups, residues, moieties, binding units or amino acid sequences may or may not provide further functionality to the immunoglobulin single variable domain, Fab, or scFv (and/or to the polypeptide in which they are present) and may or may not modify the properties of the immunoglobulin single variable domain, Fab, or scFv.

In some aspects, such further groups, residues, moieties or binding units may be one or more additional amino acids, such that the cell targeting group is a (fusion) protein or (fusion) polypeptide. In some aspects, said one or more other groups, residues, moieties or binding units are immunoglobulins. In some aspects, said one or more other groups, residues, moieties or binding units are domain antibodies (dAbs), amino acids that are suitable for use as a domain antibody, immunoglobulin single variable domains, amino acids that are suitable as immunoglobulin single variable domains, Fab, amino acids that are suitable as Fab, scFv, or amino acids that are suitable as scFv.

In some aspects, such groups, residues, moieties or binding units may for example be chemical groups, residues, moieties, which may or may not by themselves be biologically and/or pharmacologically active. In some aspects, such groups are linked to an immunoglobulin single variable domain, Fab, or scFv so as to provide a “derivative” of the immunoglobulin single variable domain, Fab, or scFv. In some aspects, said further residues may be effective in preventing or reducing binding of so-called “pre-existing antibodies” to the polypeptides. For this purpose, the polypeptides and constructs may contain a C-terminal extension (X) n in which n is 1 to 10, 1 to 5, 2 or 1; and each X is an (preferably naturally occurring) amino acid residue that is independently chosen, and preferably independently chosen from the group consisting of alanine (A), glycine (G), valine (V), leucine (L) or isoleucine (I). In some aspects, X is not cysteine.

In a cell targeting group, the one or more immunoglobulin single variable domains, Fab, or scFv and the one or more groups, residues, moieties or binding units may be linked directly to each other and/or via one or more suitable linkers. For example, when the one or more groups, residues, moieties or binding units are amino acids, the linkers may also be an amino acid, so that the resulting polypeptide is a fusion protein or fusion polypeptide.

In some aspect, a linker comprises one or more repeats of a peptide motif such as, e.g., a GS motif. In some aspects, a linker comprises a GGGGS motif (SEQ ID NO: 2) (for example, having the formula (Gly-Gly-Gly-Gly-Ser) n in which n may be 1, 2, 3, 4, 5, 6, 7 or more). In some aspects, a linker is a 9GS linker (GGGGSGGGS) (SEQ ID NO: 5), 15GS linker (SEQ ID NO: 7) (n=3) and 35GS linker (SEQ ID NO: 12) (n=7). Reference is, e.g., made to Chen et al. 2013 (Adv. Drug Deliv. Rev. 65(10): 1357-1369) and Klein et al. 2014 (Protein Eng. Des. Sel. 27 (10): 325-330). In some aspects, a linker is a 3A linker, i.e. comprising three alanines. In some aspects, a linker is a 5GS linker (SEQ ID NO: 2), a 7GS linker (SEQ ID NO: 3), a 8GS linker (SEQ ID NO: 4), a 9GS linker (SEQ ID NO: 5), a10GS linker (SEQ ID NO: 6), a 15GS linker (SEQ ID NO: 7), a 18GS linker (SEQ ID NO: 8), a 20GS linker (SEQ ID NO: 9), a 25GS linker (SEQ ID NO: 10), a 30GS linker (SEQ ID NO: 11), a 35GS linker (SEQ ID NO: 12) or a 40GS (SEQ ID NO: 13) linker as shown in TABLE 1 below.

TABLE 1

| Name | ID | Amino acid sequence |
|---|---|---|
| 3A linker | 1 | AAA |
| 5GS linker | 2 | GGGGS |
| 7GS linker | 3 | SGGSGGS |
| 8GS linker | 4 | GGGGSGGS |
| 9GS linker | 5 | GGGGSGGGS |
| 10GS linker | 6 | GGGGSGGGGS |
| 15GS linker | 7 | GGGGSGGGGSGGGGS |
| 18GS linker | 8 | GGGGSGGGGSGGGGSGGS |
| 20GS linker | 9 | GGGGSGGGGSGGGGSGGGGS |
| 25GS linker | 10 | GGGGSGGGGSGGGGSGGGGSGGGGS |
| 30GS linker | 11 | GGGGSGGGGSGGGGSGGGGSGGGGS |
| | | GGGGS |
| 35GS linker | 12 | GGGGSGGGGSGGGGSGGGGSGGGGS |
| | | GGGGSGGGGS |
| 40GS linker | 13 | GGGGSGGGGSGGGGSGGGGSGGGGS |
| | | GGGGSGGGGSGGGGS |

Cell Targeting Groups that Target Immune Cells

In some aspects, a cell targeting group or combination of targeting groups can be selected based on the desired localization, function, or structural features of a given target cell. For example, a cell targeting group can target a T cell, T cell population or T cell subpopulation. In some aspects, a cell targeting group that targets a T cell comprises one or more antibodies, immunoglobulin single variable domains, Fab, scFv, or derivatives thereof that target a T cell surface antigen. Exemplary T cell surface antigens include, but are not limited to, CD3, CD4, CD7, and CD8.

An exemplary CD3 binding agent (CD3γ/δ/ε, CD3γ, CD3δ, CD3γ/ε, CD3δ/ε, or CD3ε) can be an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), 16669-1-AP (CD3ε, Invitrogen) and antigen binding fragments thereof. In some aspects, the binding agent comprises a VH domain and a VL domain of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3ε, Invitrogen). In some aspects, the binding agent comprises the heavy chain CDR1, CDR2, and CDR3 and the light chain CDR1, CDR2, and CDR3, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196: 901-917), MacCallum (see, MacCallum R M et al., (1996) J. MOL. BIOL. 262:732-745), or any other CDR determination method known in the art, of the VH and VL sequences of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3ε, Invitrogen). An exemplary CD3 binding agent can also be selected from antibodies or antibody fragments employing CDRs of clones hsp34, OKT-3, UCHT1, 38.1, HIT3a, RFT8, SK7, BC3, SP34-2, HU291, TRX4, Catumaxomab, teplizumab, 3-106, 3-114, 3-148, 3-190, 3-271, 3-550, 4-10, 4-48, H2C, F12Q, I2C, SP7, 3F3A1, CD3-12, 301, RIV9, JB38-29, JE17-74, GT0013, 4E2, 7A4, 4D10A6, SPV-T3b, M2AB, ICO-90, 30A1 or Hu38E4.v1 (US Patent Application 20200299409A1), REGN5458 (US Patent Application 20200024356A1), or Blinatumomab

An exemplary CD4 binding agent can be an antibody selected from the group consisting of Ibalizumab (genome.jp/dbget-bin/www_bget?D09575), AF1856 (R&D Systems), MAB554 (R&D Systems), BF0174 (Affinity Biosciences), PAB31115 (Abnova Corporation), CAL4 (Abcam), and antigen binding fragments thereof. In some aspects, the binding agent comprises a VH domain and a VL domain of an antibody selected from the group consisting of AF1856 (R&D Systems), MAB554 (R&D Systems), BF0174 (Affinity Biosciences), PAB31115 (Abnova Corporation), and CAL4 (Abcam). In some aspects, the binding agent comprises the heavy chain CDR1, CDR2, and CDR3 and the light chain CDR1, CDR2, and CDR3, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the VH and VL sequences of an antibody selected from the group consisting of AF1856 (R&D Systems), MAB554 (R&D Systems), BF0174 (Affinity Biosciences), PAB31115 (Abnova Corporation), and CAL4 (Abcam). An exemplary CD4 binding agent can also be selected from antibodies or antibody fragments employing CDRs of clones Ibalizumab, OKT4, RPA-T4, S3.5, SK3, N1UG0, RIV6, OTI18E3, MEM-241, B486A1, RFT-4g, 7E14, MDX.2, MEM-115, MEM-16, ICO-86, Edu-2, or ilbalizumab.

An exemplary CD7 binding agent can be an antibody selected from the group consisting of MAB7579 (R&D Systems), AF7579 (R&D Systems), EPR22065 (Abcam), 1G10D8 (Proteintech), NBP2-32097 (Novus Biologicals), NBP2-38440 (Novus Biologicals), and antigen binding fragments thereof. In some aspects, the binding agent comprises a VH domain and a VL of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals). In some aspects, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals). An exemplary CD7 binding agent can also be selected from antibodies or antibody fragments employing CDRs of clones TH-69, 3Af11, T3-3A1, 124-1D1, 3A1f, CD7-6B7, or VHH6.

An exemplary CD8 (CD8α, CD8α/α, CD8α/β or CD8β) binding agent can be an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), YTS105.18 (CD8α, Novus Biologicals), TRX2 (US 2017-0198045 A1 or U.S. Pat. No. 10,647,768), and antigen binding fragments thereof. In some aspects, the binding agent comprises a VH domain and a VL domain of an antibody selected from the group consisting of 2.43 (Invitrogen), 51.1 (ATCC HB-230), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8a, Novus Biologicals). In some aspects, the binding agent comprises the heavy chain CDR1, CDR2, and CDR3 and the light chain CDR1, CDR2, and CDR3, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the VH and VL sequences of an antibody selected from the group consisting of 2.43 (Invitrogen), Du CD8-1 (CD8a, Invitrogen), 9358-CD (CD8a/B, R&D Systems), MAB116 (CD8α, R&D Systems), ab4055 (CD8α, Abcam), C8/144B (CD8α, Novus Biologicals), and YTS105.18 (CD8a, Novus Biologicals). An exemplary CD8 binding agent can also be selected from antibodies or antibody fragments employing CDRs of clones OKT-8, 51.1, S6F1, TRX2, and UCHT4, SP16, 3B5, C8-144B, HIT8a, RAVB3, LT8, 17D8, MEM-31, MEM-87, RIV11, DK-25, YTC141.1HL, or YTC182.20.

In some aspects, a cell targeting group targets an NK cell, or NK cell population. In some aspects, an NK cell targeting group comprises one or more antibodies, antigen binding fragments or antigen binding derivatives thereof that target an NK cell surface antigen. Exemplary NK cell surface antigens include, but are not limited to, CD7, CD48, CD56, CD85a, CD85c, CD85d, CD85e, CD85f, CD85i, CD85j, CD158b2, CD161, CD244, CD16a, CD16b, IL-2 receptor, CD27, CD28, CD48, CD69, CD70, CD86, CD112, CD122, CD155, CD161, CD244, CD266, CD314/NKG2D, CD336/NKP44, CD337/NKP30.

In some aspects, an immune cell targeting group comprises a C-terminal cysteine residue. In some aspects, an immune cell targeting group comprises a Fab that comprises a cysteine at the C-terminus of the heavy or light chain fragment. In some aspects, the Fab further comprises one or more amino acids between the heavy chain of the Fab and the C-terminal cysteine. For example, in some aspects, an Fab comprises two or more amino acids derived from an antibody hinge region (e.g., a partial hinge sequence) between the C-terminus of the Fab and the C-terminal cysteine. In some aspects, an Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment, e.g., a scFv linked to the C-terminus of the light chain constant domain by an amino acid linker.

In some aspects, an immune cell targeting group comprises an immunoglobulin single variable domain, such as an immunoglobin single variable domain (e.g., a VHH). In some aspects, the immunoglobin single variable domain comprises a cysteine at the C-terminus. In some aspects, the immunoglobin single variable domain further comprises a linker comprising one or more amino acids between the VHH domain and the C-terminal cysteine. In some aspects, the linker comprises one or more glycine residues, e.g., two glycine residues. In some aspects, the immune cell targeting group comprises two or more VHH domains. In some aspects, the two or more VHH domains are linked by an amino acid linker. In some aspects, the amino acid linker comprises one or more glycine and/or serine residues (e.g., one or more repeats of the sequence GGGGS (SEQ ID NO: 2)). In some aspects, the immune cell targeting group comprises a first VHH domain linked to an antibody CH1 domain and a second VHH domain linked to an antibody light chain constant domain, and wherein the antibody CH1 domain and the antibody light chain constant domain are linked by one or more disulfide bonds (e.g., interchain disulfide bonds). In some aspects, the immune cell targeting group comprises a VHH domain linked to an antibody CH1 domain, and wherein the antibody CH1 domain is linked to an antibody light chain constant domain by one or more disulfide bonds.

Cell Targeting Groups that Target Hematopoietic Stem Cells

In some aspects, a target cell is a hematopoietic stem cell and the cell targeting group is an antibody or antigen binding fragment thereof that binds to an antigen present on the hematopoietic stem cell.

In some aspects, a cell targeting group of a lipid nanoparticle binds to a hematopoietic stem cell surface molecule. In some aspects, the cell targeting group of the lipid nanoparticle is an antibody or fragment thereof that binds to a hematopoietic stem cell surface molecule. In some aspects, the hematopoietic stem cell molecule is CD34, CD105 (also known as endoglin), or CD117 (also known as c-kit, tyrosine-protein kinase KIT, or mast/stem cell growth factor receptor (SCFR)). In some aspects, the hematopoietic stem cell targeting group of the lipid nanoparticle is an antibody or fragment thereof that binds to CD34, CD105, and/or CD117. In some aspects, the antibody is a human or humanized antibody. In some aspects, an antibody that binds to CD105 and/or CD117 is covalently coupled to a lipid in the lipid nanoparticle via a polyethylene glycol (PEG) containing linker.

### Lipid-Cell Targeting Group Conjugates

In some aspects, a lipid nanoparticle as described herein comprises one or more lipid-cell targeting group conjugates. In some aspects, a lipid-cell targeting group conjugate comprises a cell targeting polypeptide, or fragment thereof, and a lipid. In some aspects, a cell targeting group is covalently coupled to a lipid either directly or via a linker. In some aspects, a cell targeting group is covalently coupled to a lipid via a polyethylene glycol (PEG) containing linker. In some aspects, a lipid-PEG-cell targeting group conjugate is an insertable lipid-cell targeting group conjugate of a lipid nanoparticle as described herein.

In some aspects, the lipid linked to a cell-targeting group via a PEG linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

In some aspects, a lipid-PEG-cell targeting group conjugate comprises a DSG-PEG, DSPE-PEG, DMPE-PEG, DSPG-PEG, DMG-PEG, DPPE-PEG, DPG-PEG or ceramide-PEG. In some aspects, a lipid-PEG-cell targeting group conjugate comprises a DPG-PEG.

In some aspects, the PEG in a lipid-PEG-cell targeting conjugate is PEG 1000, PEG 2000, PEG 2100, PEG 2200, PEG 2300, PEG 2400, PEG 2500, PEG 2600, PEG 2700, PEG 2800, PEG 2900, PEG 3000, PEG 3100, PEG 3200, PEG 3300, PEG 3400, PEG 3500, PEG 3600, PEG 3700, PEG 3800, PEG 3900, PEG 4000, PEG 4100, PEG 4200, PEG 4300, PEG 4400, PEG 4500, PEG 4600, PEG 4700, PEG 4800, PEG 4900, or PEG 5000. In some aspects, the PEG is PEG 2000 or PEG 3400. In some aspects, the PEG is PEG 2000 (PEG2K).

In some aspects, a lipid-cell targeting group conjugate comprises a lipid-PEG2K-antibody. In some aspects, a lipid-cell targeting group conjugate comprises a lipid-PEG2K-Fab. In some aspects, a lipid-cell targeting group conjugate comprises a lipid-PEG2K-scFv. In some aspects, a lipid-cell targeting group conjugate comprises a lipid-PEG2K-immunoglobin single variable domain (ISV domain). In some aspects, a lipid nanoparticle comprises a lipid-cell targeting group conjugate comprising a lipid-PEG2K antibody, a lipid-cell targeting group conjugate comprising a lipid-PEG2K-Fab, a lipid-cell targeting group conjugate comprising a lipid-PEG2K-scFv, and/or a lipid-cell targeting group conjugate comprising a lipid-PEG2K-ISV domain.

In some aspects, the lipid-cell targeting group conjugate comprises a DSPE-PEG2K-cell targeting polypeptide or fragment thereof. In some aspects, the lipid-cell targeting group conjugate comprises a DSPE-PEG2K-cell targeting antibody or fragment thereof. In some aspects, the lipid-cell targeting group conjugate comprises a DSPE-PEG2K-cell targeting Fab. In some aspects, the lipid-cell targeting group conjugate comprises a DSPE-PEG2K-cell targeting scFv. In some aspects, the lipid-cell targeting group conjugate comprises a DSPE-PEG2K-cell targeting ISV domain. In some aspects, a lipid nanoparticle comprises a lipid-cell targeting group conjugate comprising a DSPE-PEG2K antibody, a lipid-cell targeting group conjugate comprising a DSPE-PEG2K-Fab, a lipid-cell targeting group conjugate comprising a DSPE-PEG2K-scFv, and/or a lipid-cell targeting group conjugate comprising a DSPE-PEG2K-ISV domain.

In some aspects, the lipid-cell targeting group conjugate comprises a DSPE-PEG3.4K-cell targeting polypeptide or fragment thereof. In some aspects, the lipid-cell targeting group conjugate comprises a DSPE-PEG3.4K-cell targeting antibody or fragment thereof. In some aspects, the lipid-cell targeting group conjugate comprises a DSPE-PEG3.4K-cell targeting Fab. In some aspects, the lipid-cell targeting group conjugate comprises a DSPE-PEG3.4K-cell targeting scFv. In some aspects, the lipid-cell targeting group conjugate comprises a DSPE-PEG3.4K-cell targeting ISV domain. In some aspects, a lipid nanoparticle comprises a lipid-cell targeting group conjugate comprising a DSPE-PEG3.4K antibody, a lipid-cell targeting group conjugate comprising a DSPE-PEG3.4K-Fab, a lipid-cell targeting group conjugate comprising a DSPE-PEG3.4K-scFv, and/or a lipid-cell targeting group conjugate comprising a DSPE-PEG3.4K-ISV domain.

In some aspects, the lipid-cell targeting group conjugate comprises a DPG-PEG2K-cell targeting polypeptide or fragment thereof. In some aspects, the lipid-cell targeting group conjugate comprises a DPG-PEG2K-cell targeting antibody or fragment thereof. In some aspects, the lipid-cell targeting group conjugate comprises a DPG-PEG2K-cell targeting Fab. In some aspects, the lipid-cell targeting group conjugate comprises a DPG-PEG2K-cell targeting scFv. In some aspects, the lipid-cell targeting group conjugate comprises a DPG-PEG2K-cell targeting ISV domain. In some aspects, a lipid nanoparticle comprises a lipid-cell targeting group conjugate comprising a DPG-PEG2K antibody, a lipid-cell targeting group conjugate comprising a DPG-PEG2K-Fab, a lipid-cell targeting group conjugate comprising a DPG-PEG2K-scFv, and/or a lipid-cell targeting group conjugate comprising a DPG-PEG2K-ISV domain.

In some aspects, the lipid of a lipid-cell targeting group conjugate is conjugated to the N-terminus, C-terminus, or anywhere in the middle part of the cell targeting polypeptide or fragment thereof. In some aspects, a cell targeting polypeptide is an antibody, Fab, scFv, or immunoglobin single variable domain and the lipid is conjugated to the N-terminus of the cell targeting antibody, Fab, scFv, or immunoglobin single variable domain. In some aspects, the lipid is conjugated to the C-terminus of a cell targeting antibody, Fab, scFv, or immunoglobin single variable domain. In some aspects, the lipid is conjugated at a position between the N- and C-terminus of a cell targeting antibody, Fab, scFv, or immunoglobin single variable domain. In some aspects, the lipid of a lipid-cell targeting group conjugate is conjugated to the cell targeting polypeptide or fragment thereof through maleimide-thiol chemistry.

In some aspects, a lipid nanoparticle comprises multiple lipid-cell targeting group conjugates. In some aspects, the multiple lipid-cell targeting groups are covalently conjugated to different PEG linkers. In some aspects, the multiple lipid-cell targeting groups are presented at the surface of the lipid nanoparticle and each targeting group binds to a different molecule or portion of a molecule on the target cell. In some aspects, the multiple lipid-cell targeting groups each bind to a different epitope on a same antigen. In some aspects, the multiple lipid-cell targeting groups each bind to a different epitope on a different antigen. Advantageously, lipid nanoparticles with multiple lipid-cell targeting groups on their surface increase the avidity and specificity of targeting interactions to a particular target cell.

In some aspects, a lipid nanoparticle comprises a lipid-PEG2K-CD3 targeting group. In some aspects, a lipid nanoparticle comprises a lipid-PEG2K-CD4 targeting group. In some aspects, a lipid nanoparticle comprises a lipid-PEG2K-CD8 targeting group. In some aspects, a lipid nanoparticle comprises a lipid-cell targeting group conjugate comprising a lipid-PEG2K-CD3 targeting group, a lipid-cell targeting group conjugate comprising a lipid-PEG2K-CD4 targeting group, and/or a lipid-cell targeting group conjugate comprising a lipid-PEG2K-CD8 targeting group.

In some aspects, a lipid nanoparticle comprises a DPG-PEG2K-CD3 targeting group. In some aspects, a lipid nanoparticle comprises a DPG-PEG2K-CD4 targeting group. In some aspects, a lipid nanoparticle comprises a DPG-PEG2K-CD8 targeting group. In some aspects, a lipid nanoparticle comprises a lipid-cell targeting group conjugate comprising a DPG-PEG2K-CD3 targeting group, a lipid-cell targeting group conjugate comprising a DPG-PEG2K-CD4 targeting group, and/or a lipid-cell targeting group conjugate comprising a DPG-PEG2K-CD8 targeting group.

In some aspects, a lipid-cell targeting group conjugate comprises a DPG-PEG2K-CD3 antibody. In some aspects, a lipid-cell targeting group conjugate comprises a DPG-PEG2K-CD3 Fab. In some aspects, a lipid-cell targeting group conjugate comprises a DPG-PEG2K-CD3 scFv. In some aspects, a lipid-cell targeting group conjugate comprises a DPG-PEG2K-CD3 immunoglobin single variable domain.

In some aspects, a lipid nanoparticle comprises a lipid-cell targeting group conjugate comprising a DPG-PEG2K-CD3 antibody, a lipid-cell targeting group conjugate comprising a DPG-PEG2K-CD3 Fab, a lipid-cell targeting group conjugate comprising a DPG-PEG2K-CD3 scFv and/or a lipid-cell targeting group conjugate comprising a DPG-PEG2K-CD3 immunoglobin single variable domain.

In some aspects, a lipid-cell targeting group conjugate comprises a DPG-PEG2K-CD4 antibody. In some aspects, a lipid-cell targeting group conjugate comprises a DPG-PEG2K-CD4 Fab. In some aspects, a lipid-cell targeting group conjugate comprises a DPG-PEG2K-CD4 scFv. In some aspects, a lipid-cell targeting group conjugate comprises a DPG-PEG2K-CD4 immunoglobin single variable domain.

In some aspects, a lipid nanoparticle comprises a lipid-cell targeting group conjugate comprising a DPG-PEG2K-CD4 antibody, a lipid-cell targeting group conjugate comprising a DPG-PEG2K-CD4 Fab, a lipid-cell targeting group conjugate comprising a DPG-PEG2K-CD4 scFv, and/or a lipid-cell targeting group conjugate comprising a DPG-PEG2K-CD4 immunoglobin single variable domain.

In some aspects, a lipid-cell targeting group conjugate comprises a DPG-PEG2K-CD8 antibody. In some aspects, a lipid-cell targeting group conjugate comprises a DPG-PEG2K-CD8 Fab. In some aspects, a lipid-cell targeting group conjugate comprises a DPG-PEG2K-CD8 scFv. In some aspects, a lipid-cell targeting group conjugate comprises a DPG-PEG2K-CD8 immunoglobin single variable domain.

In some aspects, a lipid nanoparticle comprises a lipid-cell targeting group conjugate comprising a DPG-PEG2K-CD8 antibody, a lipid-cell targeting group conjugate comprising a DPG-PEG2K-CD8 Fab, a lipid-cell targeting group conjugate comprising a DPG-PEG2K-CD8 scFv, and/or a lipid-cell targeting group conjugate comprising a DPG-PEG2K-CD8 immunoglobin single variable domain.

In some aspects, a lipid nanoparticle comprises a DPG-PEG2K-CD3 antibody, a DPG-PEG2K-CD4 antibody, and/or a DPG-PEG2K-CD8 antibody. In some aspects, a lipid nanoparticle comprises a DPG-PEG2K-CD3 Fab, a DPG-PEG2K-CD4 Fab, and/or a DPG-PEG2K-CD8 Fab. In some aspects, a lipid nanoparticle comprises a DPG-PEG2K-CD3 scFv, a DPG-PEG2K-CD4 scFv, and/or a DPG-PEG2K-CD8 scFv. In some aspects, a lipid nanoparticle comprises a DPG-PEG2K-CD3 immunoglobin single variable domain, a DPG-PEG2K-CD4 immunoglobin single variable domain, and/or a DPG-PEG2K-CD8 immunoglobin single variable domain.

In some aspects, a lipid nanoparticle comprises a DPG-PEG2K-CD3 antibody, a DPG-PEG2K-CD3 Fab, a DPG-PEG2K-CD3 scFv, a DPG-PEG2K-CD3 immunoglobin single variable domain, a DPG-PEG2K-CD4 antibody, a DPG-PEG2K-CD4 Fab, a DPG-PEG2K-CD4 scFv, a DPG-PEG2K-CD4 immunoglobin single variable domain, a DPG-PEG2K-CD8 antibody, a DPG-PEG2K-CD8 Fab, a DPG-PEG2K-CD8 scFv, or a DPG-PEG2K-CD8 immunoglobin single variable domain or any combination thereof.

In some aspects, a lipid nanoparticle comprises a lipid-PEG2K-CD56 targeting group. In some aspects, a lipid-cell targeting group conjugate comprises is a lipid-PEG2K-CD56 antibody. In some aspects, a lipid-cell targeting group conjugate comprises a lipid-PEG2K-CD56 Fab. In some aspects, a lipid-cell targeting group conjugate comprises a lipid-PEG2K-CD56 scFv. In some aspects, a lipid-cell targeting group conjugate comprises a lipid-PEG2K-CD56 immunoglobin single variable domain.

In some aspects, a lipid nanoparticle comprises a DPG-PEG2K-CD56 targeting group. In some aspects, a lipid-cell targeting group conjugate comprises is a DPG-PEG2K-CD56 antibody. In some aspects, a lipid-cell targeting group conjugate comprises a DPG-PEG2K-CD56 Fab. In some aspects, a lipid-cell targeting group conjugate comprises a DPG-PEG2K-CD56 scFv. In some aspects, a lipid-cell targeting group conjugate comprises a DPG-PEG2K-CD56 immunoglobin single variable domain.

In some aspects, a lipid nanoparticle comprises a lipid-PEG2K-CD105 targeting group. In some aspects, a lipid-cell targeting group conjugate comprises a lipid-PEG2K-CD105 antibody. In some aspects, a lipid-cell targeting group conjugate comprises a lipid-PEG2K-CD105 Fab. In some aspects, a lipid-cell targeting group conjugate comprises a lipid-PEG2K-CD105 scFv. In some aspects, a lipid-cell targeting group conjugate comprises a lipid-PEG2K-CD105 immunoglobin single variable domain. In some aspects, lipid nanoparticle comprises a lipid-cell targeting group conjugate comprising a lipid-PEG2K-CD105 antibody, a lipid-cell targeting group conjugate comprising a lipid-PEG2K-CD105 Fab, a lipid-cell targeting group conjugate comprising a lipid-PEG2K-CD105 scFv, and/or a lipid-cell targeting group conjugate comprising a lipid-PEG2K-CD105 immunoglobin single variable domain.

In some aspects, a lipid nanoparticle comprises a DPG-PEG2K-CD105 targeting group. In some aspects, a lipid-cell targeting group conjugate comprises a DPG-PEG2K-CD105 antibody. In some aspects, a lipid-cell targeting group conjugate comprises a DPG-PEG2K-CD105 Fab. In some aspects, a lipid-cell targeting group conjugate comprises a DPG-PEG2K-CD105 scFv. In some aspects, a lipid-cell targeting group conjugate comprises a DPG-PEG2K-CD105 immunoglobin single variable domain. In some aspects, a lipid nanoparticle comprises a lipid-cell targeting group conjugate comprising a DPG-PEG2K-CD105 antibody, a lipid-cell targeting group conjugate comprising a DPG-PEG2K-CD105 Fab, a lipid-cell targeting group conjugate comprising a DPG-PEG2K-CD105 scFv, and/or a lipid-cell targeting group conjugate comprising a DPG-PEG2K-CD105 immunoglobin single variable domain.

In some aspects, a lipid nanoparticle comprises a lipid-PEG-CD117 targeting group. In some aspects, a lipid-cell targeting group conjugate comprises a lipid-PEG2K-CD117 antibody. In some aspects, a lipid-cell targeting group conjugate comprises a lipid-PEG2K-CD117 Fab. In some aspects, a lipid-cell targeting group conjugate comprises a lipid-PEG2K-CD117 scFv. In some aspects, a lipid-cell targeting group conjugate comprises a lipid-PEG2K-CD117 immunoglobin single variable domain. In some aspects, a lipid nanoparticle comprises a lipid-cell targeting group conjugate comprising a lipid-PEG2K-CD117 antibody, a lipid-cell targeting group conjugate comprising a lipid-PEG2K-CD117 Fab, a lipid-cell targeting group conjugate comprising a lipid-PEG2K-CD117 scFv, and/or a lipid-cell targeting group conjugate comprising a lipid-PEG2K-CD117 immunoglobin single variable domain.

In some aspects, a lipid nanoparticle comprises a DPG-PEG-CD117 targeting group. In some aspects, a lipid-cell targeting group conjugate comprises a DPG-PEG2K-CD117 antibody. In some aspects, a lipid-cell targeting group conjugate comprises a DPG-PEG2K-CD117 Fab. In some aspects, a lipid-cell targeting group conjugate comprises a DPG-PEG2K-CD117 scFv. In some aspects, a lipid-cell targeting group conjugate comprises a DPG-PEG2K-CD117 immunoglobin single variable domain. In some aspects, a lipid nanoparticle comprises a lipid-cell targeting group conjugate comprising a DPG-PEG2K-CD117 antibody, a lipid-cell targeting group conjugate comprising a DPG-PEG2K-CD117 Fab, a lipid-cell targeting group conjugate comprising a DPG-PEG2K-CD117 scFv, and/or a lipid-cell targeting group conjugate comprising a DPG-PEG2K-CD117 immunoglobin single variable domain.

In some aspects, a lipid nanoparticle comprises a lipid-PEG2K-CD34 targeting group. In some aspects, a lipid-cell targeting group conjugate comprises a lipid-PEG2K-CD34 antibody. In some aspects, a lipid-cell targeting group conjugate comprises a lipid-PEG2K-CD34 Fab. In some aspects, a lipid-cell targeting group conjugate comprises a lipid-PEG2K-CD34 scFv. In some aspects, a lipid-cell targeting group conjugate comprises a lipid-PEG2K-CD34 immunoglobin single variable domain. In some aspects, lipid nanoparticle comprises a lipid-cell targeting group conjugate comprising a lipid-PEG2K-CD34 antibody, a lipid-cell targeting group conjugate comprising a lipid-PEG2K-CD34 Fab, a lipid-cell targeting group conjugate comprising a lipid-PEG2K-CD34 scFv, and/or a lipid-cell targeting group conjugate comprising a lipid-PEG2K-CD34 immunoglobin single variable domain.

In some aspects, a lipid nanoparticle comprises a DPG-PEG2K-CD34 targeting group. In some aspects, a lipid-cell targeting group conjugate comprises a DPG-PEG2K-CD34 antibody. In some aspects, a lipid-cell targeting group conjugate comprises a DPG-PEG2K-CD34 Fab. In some aspects, a lipid-cell targeting group conjugate comprises a DPG-PEG2K-CD34 scFv. In some aspects, a lipid-cell targeting group conjugate comprises a DPG-PEG2K-CD34 immunoglobin single variable domain. In some aspects, a lipid nanoparticle comprises a lipid-cell targeting group conjugate comprising a DPG-PEG2K-CD34 antibody, a lipid-cell targeting group conjugate comprising a DPG-PEG2K-CD34 Fab, a lipid-cell targeting group conjugate comprising a DPG-PEG2K-CD34 scFv, and/or a lipid-cell targeting group conjugate comprising a DPG-PEG2K-CD34 immunoglobin single variable domain.

In some aspects, a lipid nanoparticle comprises a lipid-PEG2K-CD105 targeting group, a lipid DPG-PEG2K-CD117 targeting group, and/or a lipid-PEG2K-CD34 targeting group. In some aspects, a lipid nanoparticle comprises a DPG-PEG2K-CD105 targeting group, a DPG-PEG2K-CD117 targeting group, and/or a DPG-PEG2K-CD34 targeting group.

In some aspects, a lipid nanoparticle comprises a DPG-PEG2K-CD105 antibody, a DPG-PEG2K-CD105 Fab, a DPG-PEG2K-CD105 scFv, a DPG-PEG2K-CD105 immunoglobin single variable domain, a DPG-PEG2K-CD117 antibody, a DPG-PEG2K-CD117 Fab, a DPG-PEG2K-CD117 scFv, a DPG-PEG2K-CD117 immunoglobin single variable domain, a DPG-PEG2K-CD34 antibody, a DPG-PEG2K-CD34 Fab, a DPG-PEG2K-CD34 scFv, or a DPG-PEG2K-CD34 immunoglobin single variable domain, or any combination thereof.

In some aspects, a lipid-cell targeting group conjugate of a lipid nanoparticle comprises between about 1 g and about 50 g of cell-targeting group per mol lipid. In some aspects, the lipid-cell-targeting group conjugate of a lipid nanoparticle comprises between about 10 g and about 20 g of cell-targeting group per mol lipid. In some aspects, a lipid-antibody conjugate of a lipid nanoparticle comprises about 12 g of cell-targeting group per mol lipid.

In some aspects, a lipid-cell targeting group conjugate comprises about 1 g to about 10 g of cell targeting group per mole of lipid. In some aspects, a lipid-cell targeting group conjugate comprises about 2 g to about 9 g of cell targeting group per mole of lipid. In some aspects, a lipid-cell targeting group conjugate comprises about 3 g to about 9 g of cell targeting group per mole of lipid.

In some aspects, a lipid-antibody conjugate comprises about 1 g to about 10 g of antibody per mole of lipid. In some aspects, a lipid-antibody conjugate comprises about 2 g to about 9 g of antibody per mole of lipid. In some aspects, a lipid-antibody conjugate comprises about 3 g to about 9 g of antibody per mole of lipid.

In some aspects, a lipid-Fab conjugate comprises about 1 g to about 10 g of Fab per mole of lipid. In some aspects, a lipid-Fab conjugate comprises about 2 g to about 9 g of Fab per mole of lipid. In some aspects, a lipid-Fab conjugate comprises about 3 g to about 9 g of Fab per mole of lipid.

In some aspects, a lipid-scFv conjugate comprises about 1 g to about 10 g of scFv per mole of lipid. In some aspects, a lipid-scFv conjugate comprises about 2 g to about 9 g of scFv per mole of lipid. In some aspects, a lipid-scFv conjugate comprises about 3 g to about 9 g of scFv per mole of lipid.

In some aspects, a lipid-immunoglobin single variable domain conjugate comprises about 1 g to about 10 g of ISV domain per mole of lipid. In some aspects, a lipid-ISV domain conjugate comprises about 2 g to about 9 g of ISV domain per mole of lipid. In some aspects, a lipid-ISV domain conjugate comprises about 3 g to about 9 g of ISV domain per mole of lipid.

In some aspects, a lipid-cell targeting group conjugate is present in a lipid nanoparticle in a range of about 0.001 mol % to about 0.5 mol %. In some aspects, a lipid-cell targeting group conjugate is present in a lipid nanoparticle in a range of about 0.002 mol % to about 0.2 mol %. In some aspects, a lipid-cell targeting group conjugate is present in the lipid nanoparticle in a range of about 0.001 mol % to about 0.4 mol %; about 0.001 mol % to about 0.3 mol %; about 0.001 mol % to about 0.2 mol %; about 0.001 mol % to about 0.1 mol %; or about 0.001 mol % to about 0.08 mol %. %; about 0.001 mol % to about 0.06 mol %; about 0.001 mol % to about 0.04 mol %; about 0.001 mol % to about 0.02 mol %; or about 0.001 mol % to about 0.01 mol %. In some aspects, a lipid-cell targeting group conjugate is present in a lipid nanoparticle in a range of about 0.002 mol % to about 0.5 mol %; about 0.003 mol % to about 0.5 mol %; about 0.004 mol % to about 0.5 mol %; about 0.005 mol % to about 0.5 mol %; about 0.006 mol % to about 0.5 mol %; about 0.008 mol % to about 0.5 mol %; about 0.01 mol % to about 0.5 mol %; about 0.02 mol % to about 0.5 mol %; about 0.03 mol % to about 0.5 mol %; about 0.04 mol % to about 0.5 mol %; about 0.05 mol % to about 0.5 mol %; about 0.06 mol % to about 0.5 mol %; about 0.07 mol % to about 0.5 mol %; about 0.08 mol % to about 0.5 mol %; about 0.09 mol % to about 0.5 mol %; about 0.1 mol % to about 0.5 mol %; about 0.15 mol % to about 0.5 mol %; about 0.2 mol % to about 0.5 mol %; about 0.25 mol % to about 0.5 mol %; about 0.3 mol % to about 0.5 mol %; about 0.35 mol % to about 0.5 mol %; or about 0.4 mol % to about 0.5 mol %.

In some aspects, a lipid-cell targeting conjugate containing lipid nanoparticle further comprises free PEG lipid so as to reduce the amount of non-specific binding via the targeting group. In some aspects, the free PEG lipid can be the same or different from the PEG lipid included in the conjugate. In some aspects, the free PEG lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG), 1,2-Distearoyl-rac-glycero-3-methylpolyoxyethylene (PEG-DSG), N-palmitoyl-sphingosine-1-succinyl [methoxy (polyethylene glycol)] (PEG-ceramide), DSPE-PEG-cysteine, or a derivative thereof, all with an average PEG lengths between 2000 and 5000, or a PEG length of about 2000, 3400, or 5000. In some aspects, a lipid nanoparticle comprises a mixture of two or more PEGylated lipids.

### Cargo

In some aspects, a lipid nanoparticle comprises a cargo, e.g., a nucleic acid molecule disposed in the lipid nanoparticle for delivery to a cell (e.g., an immune cell or hematopoietic stem cell) or tissue in a subject.

In some aspects, a lipid nanoparticle comprises a nucleic acid, e.g., a DNA or RNA, such as an mRNA, tRNA, microRNA, siRNA, gRNA (guide RNA), circRNA (circular RNA), ribozymes, decoy RNA or dicer substrate siRNA. In some aspects, a nucleic acid comprises naturally occurring components, such as, naturally occurring bases, sugars or linkage groups (e.g., phosphodiester linkage groups). In some aspects, a nucleic acid comprises non-naturally occurring components or modifications, (e.g., thioester linkage groups). For example, the nucleic acid can be synthesized to contain base, sugar, linker modifications known to those skilled in the art. Furthermore, the nucleic acids can be linear or circular, or have any desired configuration. In some aspects, a lipid nanoparticle includes multiple nucleic acid molecules, e.g., multiple RNA molecules, which can be the same or different.

In some aspects, one or more lipid nanoparticle compositions including one or more different mRNAs are combined, and/or simultaneously contacted with a cell. In some aspects, an mRNA may include one or more of a stem loop, a chain terminating nucleoside, a polyA sequence, a polyadenylation signal, and/or a 5′ cap structure. In some aspects, the mRNA may encode a receptor, such as a chimeric antigen receptor (CAR), for transfection of an immune cell. In some aspects, the mRNA may encode a Cas nuclease. In some aspects, the RNA may comprise a guide RNA.

In some aspects, the wt/wt ratio of the lipid components to the cargo (e.g., mRNA) in the lipid nanoparticle is from about 1:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, or 50:1. In some aspects, the wt/wt ratio of the lipid components to the cargo in the lipid nanoparticle is from about 5:1 to about 50:1. In some aspects, the wt/wt ratio of the lipid components to the cargo in the lipid nanoparticle is from about 5:1 to about 40:1. In some aspects, the wt/wt ratio of the lipid components to the cargo in the lipid nanoparticle is from about 10:1 to about 40:1. In some aspects, the wt/wt ratio of the lipid components to the cargo in the lipid nanoparticle is from about 15:1 to about 25:1.

In some aspects, the encapsulation efficiency of the cargo (e.g., mRNA) in the lipid nanoparticle is at least 50%, at least 55%, at least 60%, at least 65%, at least 70%, at least 75%, at least 80%, at least 85%, at least 90%, at least 95%, at least 97% or 99%.

In some aspects, a lipid nanoparticle as described herein exhibits dye accessible RNA of less than 50%, less than 45%, less than 40%, less than 35%, less than 30%, less than 25%, less than 20%, less than 15%, less than 10%, less than 7.5%, less than 5%, less than 2.5%, or less than 1%.

In some aspects, the one or more mRNAs and lipids are selected to provide a specific N:P ratio (the ratio of positively-chargeable lipid or polymer amine (N=nitrogen) groups to negatively-charged nucleic acid phosphate (P) groups). The N:P ratio of a lipid nanoparticle composition refers to the molar ratio of nitrogen atoms in one or more lipids to the number of phosphate groups in an mRNA. In general, a lower N:P ratio is preferred. A N:P ratio may be dependent on a specific lipid and its pKa. In some aspects, the mRNA and lipids in a lipid nanoparticle and/or their relative amounts are selected to provide an N:P ratio from about 1:1 to about 30:1, or from about 1:1 to about 20:1. In some aspects, the N:P ratio is, e.g., 1:1, 2:1, 3:1, 4:1, 5:1, 6:1, 7:1, or 8:1. In certain aspects, the N:P ratio is from about 2:1 to about 5:1. In some aspects, the N:P ratio is about 4:1. In some aspects, the N:P ratio is from about 4:1 to about 8:1. In some aspects, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

### Compositions

Provided herein are compositions comprising a lipid nanoparticle described herein. In some aspects, a composition provided herein comprises a lipid nanoparticle comprising an ionizable lipid. In some aspects, an ionizable lipid of the lipid nanoparticle comprises a structure of Formula I:

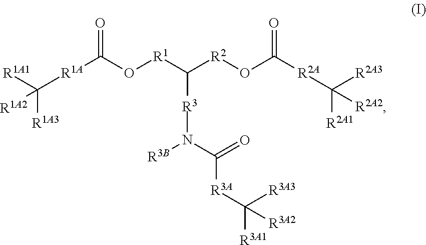

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂) 0-10C(O)R^{a2}; R^{a1} and R^{a2} are each independently C₁₋₂₀ alkyl or C₁₋₂₀ alkenyl; R^{3B} is

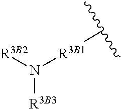

R^{3B1} is C₁₋₆ alkylene; and
R^{3B2} and R^{3B3} are each independently H or C₁₋₆ alkyl.

In some aspects, an ionizable lipid of the lipid nanoparticle comprises a structure of Formula II (Lipid 15):

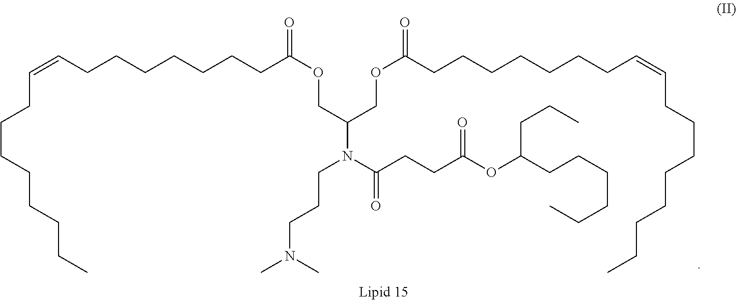 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In some aspects, an ionizable lipid of the lipid nanoparticle comprises a structure of Formula III (KC2):

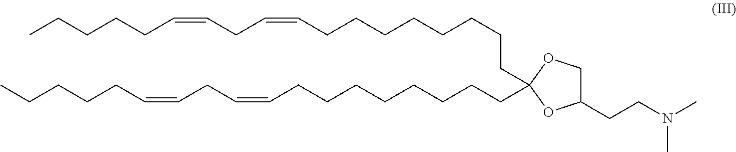 [SMILES: CCCCC/C=C\C/C=C\CCCCCCCCC1(CCCCCCCC/C=C\C/C=C\CCCCC)OCC(CCN(C)C)O1]

In some aspects, an ionizable lipid of the lipid nanoparticle comprises a structure of Formula IV (KC3):

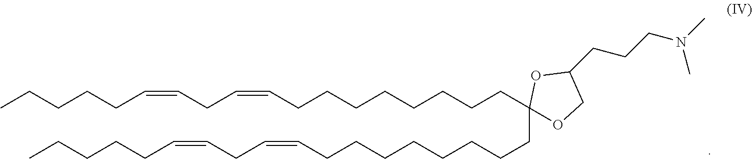 [SMILES: CCCCC/C=C\C/C=C\CCCCCCCCC1(CCCCCCCC/C=C\C/C=C\CCCCC)OCC(CCCN(C)C)O1]

In some aspects, the ionizable lipid of the lipid nanoparticle comprises a structure of Formula V:

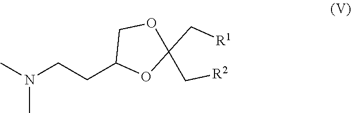

wherein R¹ and R² are each independently a lipid.

In some aspects, the ionizable lipid of the lipid nanoparticle comprises a structure of Formula VI:

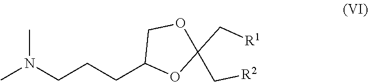

wherein R¹ and R² are each independently a lipid.

In some embodiments, where the ionizable lipid of the lipid nanoparticle comprises a structure of Formula V or Formula VI, R¹ and R² are each independently substituted or unsubstituted alkyl, substituted or unsubstituted alkenyl. In some embodiments, where the ionizable lipid of the lipid nanoparticle comprises a structure of Formula V or Formula VI, R¹ and R² are the same lipid. In some embodiments, where the ionizable lipid of the lipid nanoparticle comprises a structure of Formula V or Formula VI, R¹ and R² are different lipids. In some embodiments, the lipid is a hydrocarbon (e.g., substituted or unsubstituted, saturated or unsaturated, branched or unbranched hydrocarbon). A hydrocarbon may be an alkane, alkene, or alkyne. In some embodiments, the hydrocarbon chain is saturated or unsaturated. In certain embodiments, an unsaturated hydrocarbon chain comprises at least one, at least one two, at least one three, at least one four, at least one five, or at least one six carbon-carbon double bonds (e.g., cis double bonds and/or trans double bonds). In some embodiments, the lipid is substituted or unsubstituted C₇₋₃₆ alkyl. In certain embodiments, a lipid is substituted or unsubstituted C₇₋₃₆ alkenyl. In certain embodiments, the lipid is unsubstituted C₇₋₃₆ alkyl or unsubstituted C₇₋₃₆ alkenyl. In certain embodiments, a hydrocarbon is substituted with alkyl, alkenyl, alkynyl, amino, alkylamino, dialkylamino, trialkylamino, hydroxyl, alkoxy, carbonyl, halogen, aryl, heterocyclic, aromatic heterocyclic, cyano, amide, carbamoyl, carboxylic acid, ester, thioether, alkylthioether, thiol, ureido, or a combination thereof. Each of these groups may in turn be substituted. In some embodiments, a hydrocarbon is substituted with one, two, three, four, five, six, seven, eight, nine, ten, or more than ten substituents.

In some aspects, the lipid nanoparticle of the composition further comprises a helper lipid, a structural lipid, a PEG lipid and, optionally, a cargo. In some aspects, the lipid nanoparticle of the composition further comprises a lipid-cell targeting group conjugate as described herein.

In some aspects, the lipid nanoparticle composition comprises one or more pharmaceutically acceptable excipient. In some aspects, the lipid nanoparticle composition comprises one or more solvent, one or more salt, and/or one or more sugar or other cryoprotectant. In some aspects, conventional excipients and accessory ingredients are used in a pharmaceutical composition, except insofar as any conventional excipient or accessory ingredient may be incompatible with one or more components of a lipid nanoparticle composition described herein. For example, an excipient or accessory ingredient may be incompatible with a component of a lipid nanoparticle composition if its combination with the component may result in any undesirable biological effect or otherwise deleterious effect.

In some aspects, one or more excipients may make up greater than 50% of the total mass or volume of a pharmaceutical composition including a lipid nanoparticle. For example, the one or more excipients may make up 30%, 40%, 50%, 60%, 70%, 80%, 90%, or more of a pharmaceutical composition.

### Methods

### Methods of Making Lipid Nanoparticles

In some aspects, lipid nanoparticles are produced by using either rapid mixing by an orbital vortexer, a T-mixer or by microfluidic mixing. Orbital vortexer mixing is accomplished by rapid addition of a lipid solution in ethanol to an aqueous solution of a nucleic acid followed immediately by vortexing at 2,500 rpm. In some aspects, a lipid nanoparticle is produced using a microfluidic mixing step. In some aspects, microfluidic mixing is achieved mixing aqueous and organic streams at a controlled flow rate in a microfluidic channel using, e.g., a NanoAssemblr device and microfluidic chips featuring optimized mixing chamber geometry (Precision Nanosystems, Vancouver, BC). In some aspects, a lipid nanoparticle is produced using a microfluidic mixing step to rapidly mix an ethanolic lipid solution and an aqueous nucleic acid solution, resulting in encapsulation of the nucleic acid in the solid lipid nanoparticles. The nanoparticle suspension is then buffer exchanged into an all aqueous buffer using membrane filtration device of choice for ethanol removal and nanoparticle maturation

### Methods of Use

The present disclosure provides methods of delivering a cargo to a target cell or tissue, e.g., a target cell or tissue in a subject, and lipid nanoparticles or pharmaceutical compositions containing the lipid nanoparticles for use in such methods. Any disclosure herein of a method of, e.g., treating a disease or disorder or, e.g., delivering a nucleic acid to a cell or, e.g., producing a polypeptide of interest in a cell should be interpreted also as a disclosure of a lipid nanoparticle or pharmaceutical composition comprising said lipid nanoparticle for use in such methods.

In some aspects, provided is a method of producing a polypeptide of interest in a target cell, and lipid nanoparticles or pharmaceutical compositions containing the lipid nanoparticle for use in such methods. Methods of producing polypeptides in such a cell involve contacting a cell with a lipid nanoparticle comprising a cargo, e.g., a nucleic acid of interest. Upon contacting the cell with the lipid nanoparticle, the nucleic acid may be taken up and translated in the cell to produce the polypeptide of interest.

In some aspects, the step of contacting a cell with a lipid nanoparticle composition including a cargo, e.g., a mRNA encoding a polypeptide of interest may be performed in vivo, ex vivo, or in vitro. The amount of a lipid nanoparticle composition contacted with a cell, and/or the amount of mRNA therein, may depend on the type of cell or tissue being contacted, the means of administration, the physiochemical characteristics of the lipid nanoparticle and the mRNA (e.g., size, charge, and chemical composition) therein, and other factors.

In some aspects, the step of contacting a lipid nanoparticle including a cargo with a cell involves or causes transfection where the lipid nanoparticle fuses with the membrane of the cell to permit the delivery of the cargo into the cell. Upon introduction into the cytoplasm of the cell, the cargo, e.g., a mRNA, is then translated into a protein or peptide via the protein synthesis machinery within the cytoplasm of the cell.

In some aspects, the efficiency of polypeptide production in the cell may be determined, and the cell may be re-contacted with a lipid nanoparticle repeatedly until a desired target polypeptide production efficiency is achieved.

In some aspects, provided are methods of delivering a cargo, e.g., a nucleic acid to a mammalian cell or tissue, e.g., a mammalian cell or tissue in a subject. In some aspects, the delivery of a nucleic acid to such a cell or tissue involves administering a lipid nanoparticle composition described herein to a subject, e.g., by injection into the subject.

In some aspects, after administration, the lipid nanoparticle can target and/or contact a cell, e.g., an immune cell or a hematopoietic stem cell in the subject. Upon contacting the cell with the lipid nanoparticle composition, a nucleic acid, e.g., a translatable mRNA, may be translated in the cell to produce a polypeptide of interest.

In some aspects, a lipid nanoparticle described herein provides at least one of the following benefits:
a. increased specificity of targeted delivery to the target cell compared to a reference lipid nanoparticle;
b. increased half-life of the nucleic acid or a polypeptide encoded by the nucleic acid in the target cell compared to a reference lipid nanoparticle;
c. increased transfection rate compared to a reference lipid nanoparticle; and/or
d. high nucleic acid encapsulation efficiency.

In some aspects, no more than 1%, 2%, 3%, 4%, 5%, 6%, 7%, 8%, 9%, or 10% of non-target cells are transfected by a lipid nanoparticle described herein. For example, in some aspects, no more than 1%, 2%, 3%, 4%, 5%, 6%, 7%, 8%, 9%, or 10% of undesired immune cells that are not meant to be the destination of the delivery are transfected by the lipid nanoparticle. In some aspects, no more than 1%, 2%, 3%, 4%, 5%, 6%, 7%, 8%, 9%, or 10% of undesired hematopoietic stem cells that are not meant to be the destination of the delivery are transfected by the lipid nanoparticle.

In some aspects, the half-life of a nucleic acid delivered by a lipid nanoparticle described herein to a target cell, e.g., an immune cell or hematopoietic stem cell, or of a polypeptide encoded by the nucleic acid delivered by the lipid nanoparticle is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of the nucleic acid delivered by a reference lipid nanoparticle to said target cell or a polypeptide encoded by the nucleic acid delivered by the reference lipid nanoparticle.

In some aspects, the composition of the lipid nanoparticle differs from the composition of a reference lipid nanoparticle in the type of ionizable cationic lipid, relative amount of ionizable cationic lipid, type of PEG lipid, relative amount of PEG lipid, type of helper lipid, relative amount of helper lipid, type of structural lipid, relative amount of structural lipid, cargo, or type of cell targeting group, or any combination thereof. In some aspects, a reference lipid nanoparticle is a lipid nanoparticle that comprises a helper lipid in a range of less than 20 mol %. In some aspects, a reference lipid nanoparticle is a lipid nanoparticle that comprises a helper lipid in a range of less than 16 mol %. In some aspects, a reference lipid nanoparticle is a lipid nanoparticle that comprises a helper lipid in a range of about 10 mol % to about 15 mol %. In some aspects, a reference lipid nanoparticle is a lipid nanoparticle that comprises a helper lipid in a range of more than 50 mol %.

In some aspects, the composition of the lipid nanoparticle differs from the composition of the reference lipid nanoparticle only in the type of ionizable cationic lipid. In some aspects, the composition of the lipid nanoparticle differs from the composition of the reference lipid nanoparticle in the relative amount of an ionizable cationic lipid. In some aspects, the composition of the lipid nanoparticle differs from the composition of the reference lipid nanoparticle only in the type of helper lipid. In some aspects, the composition of the lipid nanoparticle differs from the composition of the reference lipid nanoparticle in the relative amount of a helper lipid. In some aspects, the composition of the lipid nanoparticle differs from the composition of the reference lipid nanoparticle in the relative amount of helper lipid where the lipid nanoparticle comprises about 16 mol % to about 20 mol % of helper lipid, while the reference lipid nanoparticle comprises about 10 mol % to about 15 mol % of the helper lipid and the difference in overall lipid content is made up by changes in the amount of structural lipid in the lipid nanoparticle; otherwise the lipid nanoparticle and the reference lipid nanoparticle are the same.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more target cells that are meant to be the destination of the delivery are transfected by the lipid nanoparticle.

In some aspects, expression level of a nucleic acid in a target cell delivered by a lipid nanoparticle described herein is at least 5%, 10%, 15%, 20%, 25%, 30%, 40%, 50%, 60%, 70%, 80%, 90%, 100%, 1.5 times, 2 times, 3 times, 4 times, 5 times, 10 times, 15 times, 20 times or more higher than expression level of the nucleic acid in the same target cell delivered by a reference lipid nanoparticle.

In some aspects, specific delivery using lipid nanoparticles described herein may result in a greater than 2 fold, 5 fold, 10 fold, 15 fold, or 20 fold increase in the amount of cargo delivered to the targeted destination as compared to another destinations (e.g., a non-target cell).

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune target cells are transfected by the lipid nanoparticle.

In some aspects, expression level of a nucleic acid in an immune target cell delivered by a lipid nanoparticle described herein is at least 5%, 10%, 15%, 20%, 25%, 30%, 40%, 50%, 60%, 70%, 80%, 90%, 100%, 1.5 times, 2 times, 3 times, 4 times, 5 times, 10 times, 15 times, 20 times or more higher than expression level of the nucleic acid in an immune target cell delivered by a reference lipid nanoparticle.

In some aspects, specific delivery using lipid nanoparticles described herein may result in a greater than 2 fold, 5 fold, 10 fold, 15 fold, or 20 fold increase in the amount of cargo delivered to an immune target cell as compared to an immune non-target cell or other non-target cell.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more hematopoietic stem cell target cells are transfected by the lipid nanoparticle.

In some aspects, expression level of a nucleic acid in a hematopoietic stem cell delivered by a lipid nanoparticle described herein is at least 5%, 10%, 15%, 20%, 25%, 30%, 40%, 50%, 60%, 70%, 80%, 90%, 100%, 1.5 times, 2 times, 3 times, 4 times, 5 times, 10 times, 15 times, 20 times or more higher than expression level of the nucleic acid in the same hematopoietic stem cell delivered by a reference lipid nanoparticle.

In some aspects, specific delivery using lipid nanoparticles described herein may result in a greater than 2 fold, 5 fold, 10 fold, 15 fold, or 20 fold increase in the amount of cargo delivered to a hematopoietic stem target cell as compared to a hematopoietic non-target cell or other non-target cell.

In some aspects, a lipid nanoparticle composition described herein may be useful for treating a disease, disorder, or condition characterized by a missing or aberrant polypeptide or polypeptide activity. In some aspects, upon delivery of a lipid nanoparticle comprising a nucleic acid, e.g., a mRNA encoding the missing or aberrant polypeptide to a cell, translation of the mRNA may produce the polypeptide, thereby reducing or eliminating an issue caused by the absence of or aberrant activity caused by the polypeptide.

In some aspects, provided herein are methods of targeting the delivery of a nucleic acid to an immune cell of a subject. In some aspects, the method comprises contacting the immune cell with a lipid nanoparticle as described herein. In some aspects, the method comprises contacting the immune cell in vitro with a lipid nanoparticle. In some aspects, the method comprises contacting the immune cell ex vivo with a lipid nanoparticle. In some aspects, the method comprises contacting the immune cell in vivo with a lipid nanoparticle.

In some aspects, provided are methods of modulating cellular function of an immune cell of a subject. In some aspects, the method comprises administering to the subject a lipid nanoparticle. In some aspects, the lipid nanoparticle comprises an immune cell targeting group.

In some aspects, the immune cell targeting group is a CD3 targeting group, a CD4 targeting group, a CD7 targeting group, or a CD8 targeting group.

In some aspects, the modulation of cell function comprises reprogramming an immune cell to initiate an immune response. In some aspects, the modulation of cell function comprises expressing a chimeric antigen receptor (CAR) in an immune cell. In some aspects, the modulation of cell function comprises contacting a T cell with a lipid nanoparticle that comprises a RNA encoding a CAR. In some aspects, the lipid nanoparticle further comprises a T cell targeting group, e.g., a CD3 targeting group, a CD4 targeting group and/or a CD8 targeting group. In some aspects, the modulation of cell function comprises increasing cytotoxic activity in a CD8+ T cell, the lipid nanoparticle used to contact the T cell comprises a CD8 targeting group and the nucleic acid disposed in the lipid nanoparticle encodes a CAR.

In some aspects, the modulation of cell function comprises delivering a nucleic acid encoding a gene editing system (e.g., a site-directed nuclease and, optionally, comprising a guide RNA) to a T cell.

In some aspects, the immune cell targeting group is CD56. In some aspects, the modulation of cell function comprises reprogramming an NK cell to initiate an immune response. In some aspects, the modulation of cell function comprises expressing a CAR in an NK cell. In some aspects, the modulation of cell function comprises increasing cytotoxic activity in an NK cell.

In some aspects, the modulation of cell function comprises delivering a nucleic acid encoding a gene editing system (e.g., a site-directed nuclease and, optionally, a guide RNA) to an NK cell.

In some aspects, provided herein are methods of targeting the delivery of a nucleic acid to a hematopoietic stem cell of a subject. In some aspects, the method comprises contacting the hematopoietic stem cell with a lipid nanoparticle as described herein. In some aspects, the method comprises contacting the hematopoietic stem in vitro with a lipid nanoparticle. In some aspects, the method comprises contacting the hematopoietic stem cell ex vivo with a lipid nanoparticle. In some aspects, the method comprises contacting the hematopoietic stem cell in vivo with a lipid nanoparticle.

In some aspects, the lipid nanoparticle comprises a hematopoietic stem cell targeting group. In some aspects, the hematopoietic stem cell targeting group is a CD105 targeting group, a CD117 targeting group, or a CD34 targeting group.

In some aspects, a lipid nanoparticle provided herein is delivered to a subject with a disease for in vivo gene editing and treatment of the disease. In some aspects, the lipid nanoparticle is delivered to a subject with a hemoglobinopathy. In some aspects, provided is a method of treating an α-hemoglobinopathy. In some aspects, provided is a method of treating a β-hemoglobinopathy. In some aspects, the β-hemoglobinopathy is a β-thalassemia. In some aspects, the β-hemoglobinopathy is sickle cell disease.

In some aspects, a lipid nanoparticle as described herein is delivered to a subject with sickle cell disease or beta-thalassemia for in vivo gene editing and treatment of the subject. In some aspects, the lipid nanoparticle comprises one or more nucleic acids encoding a gene editing system targeting a locus wherein gene editing results in increased hemoglobin F (HbF) in a cell of a subject for the treatment of a disease (e.g., sickle cell disease and beta-thalassemia).

In some aspects, the lipid nanoparticle that is delivered to a subject comprises one or more nucleic acids encoding a site-directed nuclease and one or more RNAs that confer binding of the Cas nuclease to the target nucleotide sequence. In some aspects, the one or more RNAs conferring binding of a Cas nuclease to the target nucleotide sequence include a transactivating cRNA (tracrRNA) and a CRISPR RNA (crRNA), or, more commonly, a guide RNA (also referred to as a single guide RNA (sgRNA)), in which crRNA and tracrRNA are engineered into one RNA molecule. In some aspects, the one or more nucleic acids encoding a nuclease encode a site-directed nuclease including a CRISPR-associated (Cas) nuclease, a zinc finger nuclease (ZEN), a transcription activator-like effector nuclease (TALEN), or a megaTAL.

### Uses of the Lipid Nanoparticles

In some aspects, the disclosure provides a lipid nanoparticle or a composition containing thereof, as disclosed herein, for use in a method of targeting the delivery of a nucleic acid to a target cell of a subject. In some aspects, the disclosure provides a lipid nanoparticle or a composition containing thereof, as disclosed herein, for use in a method of targeting the delivery of a nucleic acid to an immune cell of a subject. In some aspects, the nucleic acid encodes an immune cell function enhancing polypeptide and such nucleic acid is targeted to an immune cell by the lipid nanoparticle. In some aspects, the lipid nanoparticle comprises an immune cell targeting group to target the delivery of the nucleic acid to the immune cell.

In some aspects, the disclosure provides a lipid nanoparticle or a composition containing thereof, as disclosed herein, for use in a method of targeting the delivery of a nucleic acid to a hematopoietic stem cell of a subject. In some aspects, the nucleic acid of the lipid nanoparticle encodes a polypeptide that functions in gene editing and such nucleic acid is targeted to a hematopoietic stem cell by the lipid nanoparticle. In some aspects, the lipid nanoparticle comprises a hematopoietic stem cell targeting group to target the delivery of the nucleic acid to a hematopoietic stem cell.

In some aspects, the disclosure provides a lipid nanoparticle or a composition containing thereof, as disclosed herein, for use in a method of treating a disease or condition in a subject. In some aspects, the lipid nanoparticle comprises a nucleic acid that encodes an immune cell function enhancing polypeptide such as, e.g., a CAR, and the targeted immune cell expressing the CAR treats a disease or condition in the subject. In some aspects, the lipid nanoparticle comprises a nucleic acid that provides components of a gene editing system and the targeted hematopoietic stem cell after having undergone gene editing treats a disease or condition in the subject. In some aspects, the composition used in the method is a pharmaceutical composition.

### Kits

In some aspects, provided is a kit for treating a disorder. In some aspects, a kit comprises: an ionizable cationic lipid, a helper lipid, a structural lipid, a PEG lipid, a cargo, a lipid-immune cell targeting group conjugate, and/or a lipid-hematopoietic stem cell targeting group conjugate, and instructions for preparing a lipid nanoparticle and treating a medical disorder using a lipid nanoparticle. In some aspects, a kit comprises a lipid nanoparticle composition comprising a lipid nanoparticle comprising an ionizable cationic lipid, a helper lipid, a structural lipid, a PEG lipid, a cargo, a lipid-immune cell targeting group conjugate and/or a lipid-hematopoietic stem cell targeting group conjugate and instructions for treating a medical disorder using the lipid nanoparticle composition or lipid nanoparticle.

### EXAMPLES

### Example 1: Preparation of Lipid Nanoparticles

Lipid nanoparticles (LNPs) encapsulating an mRNA cargo were prepared by mixing an aqueous mRNA solution and an ethanolic lipid blend solution (containing ionizable lipid, DSPC, DPG-PEG and Cholesterol at lipid ratios shown in TABLE 2) using an in-line microfluidic mixing process. The mRNA cargo used for immune cell targeting LNPs was eGFP mRNA and the mRNA cargo used for HSC targeting LNPs was mCherry mRNA. Several lipid nanoparticles were produced with different amounts of DSPC, e.g., DSPC of 10 mol %, 12.5 mol %, 15 mol %, 20 mol %, 30 mol %, or 40 mol %.

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were mounted into the luer ports of the mixing cartridge. The two solutions were then mixed at a 3:1 v/v ratio of mRNA solution (1.5 mL) to lipid solution (0.5 mL) at a total flow rate of 9 mL/min using the NanoAssemblr Ignite. The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with HBS exchange buffer (25 mM pH 7.4 HEPES buffer with 150 mM NaCl). The LNP suspension (2 mL) was then loaded into the device and centrifuged at 500 RCF until the volume was reduced by half volume (1 mL). The suspension was then diluted with exchange buffer (1 mL, 25 mM pH 7.4 HEPES buffer) to bring the suspension back to the original volume. This process of two-fold concentration and two-fold dilution was repeated five additional times for a total of six discontinuous diafiltration steps. The LNP suspension was then exchanged into MBS (25 mM pH 6.5 MES buffer with 150 mL NaCl) by diluting ten-fold with MBS and centrifuging at 500 RCF until the volume was reduced by one tenth. This ten-fold dilution with MBS and ten-fold concentration step was repeated one more time. The retentate containing the LNPs in MBS was recovered from the centrifugal ultrafiltration device and stored at 4° C. until further use.

### Example 2: Characterization of LNPs

Samples of the LNPs produced in Example 1 were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer. The results for Formulation 1 and 2 LNPs are shown in TABLE 2.

TABLE 2

| | | | | | | DLS Z- | | Zeta | |
|---|---|---|---|---|---|---|---|---|---|
| | | | | | | Avg. | | Potential | |
| | | Lipid | | Fab | mRNA | Diameter | DLS | at pH 7.4 | Endotoxin |
| No. | Lipids | mol % | N/P | density | (μg/ml) | (nm) | PDI | (mV) | (EU/ml) |
| 1 | Lipid | 49.24/39.4/ | 4.81 | 12 | 150 | 80 | 0.1 | 0.1 | <0.4 |
| | 15/Chol/DSPC/ | 9.85/1.51 | | | | | | | |
| | DPG-PEG2K | | | | | | | | |
| 2 | Lipid | 49.24/29.25/ | 4.81 | 12 | 150 | 80 | 0.09 | −1.4 | <0.4 |
| | 15/Chol/DSPC/ | 20/1.51 | | | | | | | |
| | DPG-PEG2K | | | | | | | | |

### Example 3: Preparation of Conjugates to Enable T Cell and Hematopoietic Stem Cell Targeting

VHHs that bind to T cell-specific (CD8) and HSC-specific targets (CD117) were conjugated to DSPE-PEG (3.4K)-maleimide and DSPE-PEG (2k)-maleimide, respectively, via covalent coupling between the maleimide group and a C-terminal cysteine in the heavy chain (HC). In a representative reaction, the protein solution in PBS with 5 mM EDTA was concentrated to 3-4 mg/mL using a 10 kDa MWCO regenerated cellulose centrifugal filter. C-terminal cysteine residues were selectively reduced using 2-5 mM TCEP at room temperature for 1.5 hours. TCEP was removed using a 7K MWCO spin desalting column. The reduced protein was mixed with a mixture of DSPE-PEG-Mal and DSPE-PEG-OMe at a 1:1 molar ratio of DSPE-PEG-Mal to Fab or VHH and a 1:4 molar ratio of DSPE-PEG-Mal:DSPE-PEG-OMe. The reaction mixture was incubated at 37° C. for 2 hours and then quenched using 15 mM cysteine at 37° C. for 15 min. The resulting DSPE-PEG-Fab or DSPE-PEG-VHH micelles were then concentrated and buffer-exchanged into pH 7.4 HEPES buffered saline using a 50K MWCO regenerated cellulose centrifugal filter. The concentration/buffer exchange process also removes residual cystine and unreacted Fab or VHH. The material was characterized by SDS-PAGE and HPLC. Fab or VHH concentration was determined by measuring the absorbance at 280 nm.

### Example 4: Human T Cell Transfection

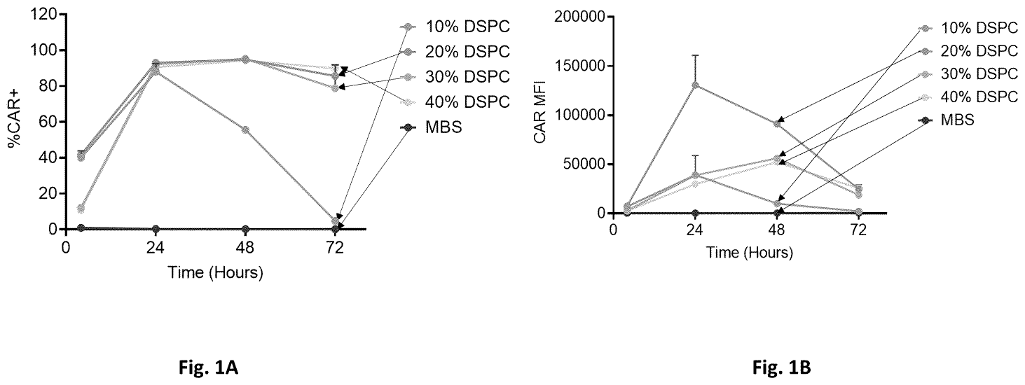

Human CD8+ T cells were isolated from PBMCs of healthy donors and cultured with human IL-2 for 24 hours (day 0). LNPs containing 10 mol %, 12.5 mol %, 15 mol %, 20 mol %, 30 mol % or 40 mol % DSPC helper lipid, respectively, CAR mRNA and GFP mRNA cargo were used for T cell transfection. On day 1, CD8+ cells were transfected with LNPs at increasing concentrations (0.012 ug/ml, 0.037 ug/ml, 0.11 ug/ml, 0.33 ug/ml, 1 μg/ml, or 3 μg/ml). Transfected cells were washed after 4 hours, stained, and analyzed immediately (0 hours) or after 24, 48, and 72 hours to determine the percentage of CAR positive cells and CAR mean fluorescence intensity. Results of T cells transfected with 0.33 ug/ml LNPs containing between 10 mol % and 40 mol % DSPC are shown in FIG. 1. T cells transfected with LNPs containing 20 mol %, 30 mol % or 40 mol % DSPC demonstrated lasting transfection efficiencies with more than 80% of T cells expressing the CAR over the 72 hours measurement period (FIG. 1A). In contrast, T cells transfected with LNPs containing 10 mol % DSPC were gradually lost over the 72 hour period (FIG. 1A). Further, T cells transfected with LNPs containing 20 mol % DSPC demonstrated substantially higher mean fluorescence intensity (MFI) than T cell transfected with LNPs containing 10 mol %, 30 mol % or 40 mol % DSPC (FIG. 1B). These results demonstrated the high transfection efficacy and low toxicity of LNPs containing 20 mol % DSPC.

### Example 5: Efficacy of Target Cell Killing by LNP-Treated T Cells

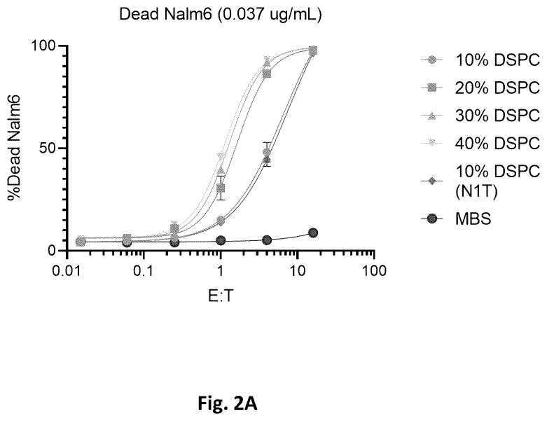

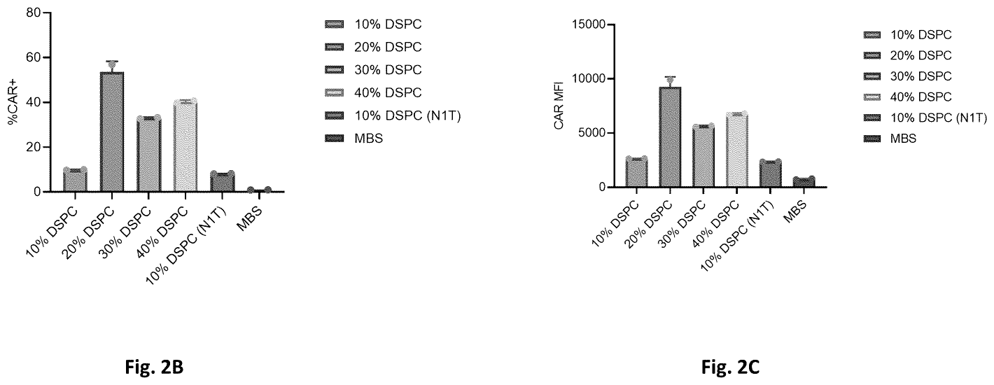

Human T effector cells transfected with 0.037 ug/ml LNPs containing 10 mol % to 40 mol % DSPC were incubated with Nalm6 target cells at E:T ratios of 0.01 to 20 and the percentage of dead Nalm6 target cells was determined. Effector T cells transfected with LNPs containing 20 mol %, 30 mol %, and 40 mol % DSPC killed more Nalm6 target cells than effector T cells transfected with LNPs containing 10 mol % DSPC (FIG. 2A). Within the effector T cells, more than 50% of cells expressed a CAR when transfected with LNPs containing 20 mol % DSPC compared to only 10% of T effector cells transfected with LNPs containing 10 mol % DSPC. About 30% and about 40% of effector T cells transfected with LNPs containing 30 mol % and 40 mol % DSPC expressed a CAR (FIG. 2B). Furthermore, CAR mean fluorescence intensity (MFI) was highest in effector T cells transfected with LNPs containing 20 mol % DSPC compared to T cells transfected with LNPs containing 10 mol %, 30 mol % or 40 mol % DSPC (FIG. 2C).

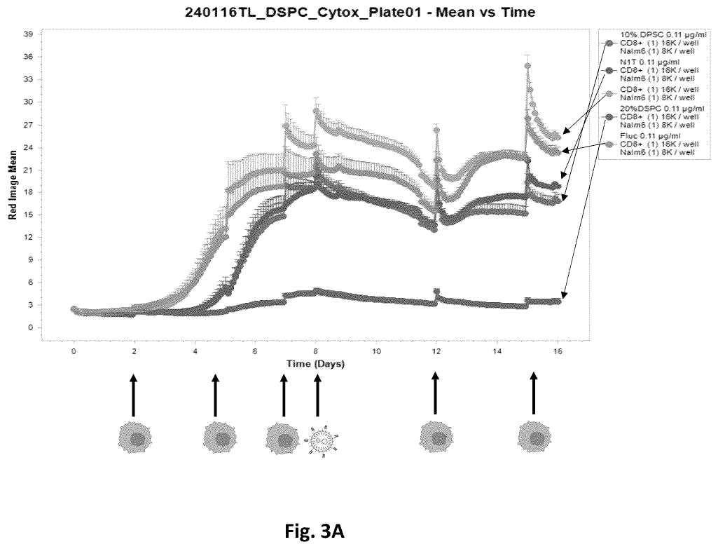

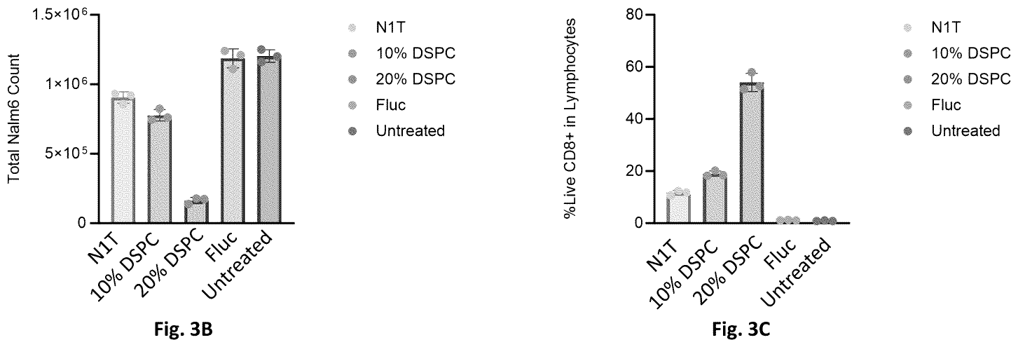

Effector cell function was measured over a 16-day period. Effector T cells transfected with LNPs containing 10 mol % to 40 mol % DSPC were co-cultured with Nalm6 target cells expressing a red fluorescence protein at an E:T ratio of 2:1 and red fluorescence was determined over the 16-day period. Fresh Nalm6 target cells were added to the co-culture every 2 to 3 days and LNPs were added to the co-culture on day 8. Only effector T cells transfected with LNPs containing 20 mol % DSPC effectively removed Nalm6 target cells over the 16 day period (FIGS. 3A and 3B). In contrast, effector T cells transfected with LNPs containing 10 mol % DSPC only slightly reduced the numbers of Nalm6 targets (FIGS. 3A and 3B). Furthermore, in the group transfected with 20 mol % DSPC LNPs, about 55% of lymphocytes were live CD8+ effector T cells at day 16 compared to less than 20% in the group transfected with 10 mol % DSPC LNPs indicating prolonged target engagement, proliferation, and survival in the 20 mol % DSPC LNP group (FIG. 3C).

### Example 6: Efficacy of Targeting T Cells of Different Human Donors by LNP

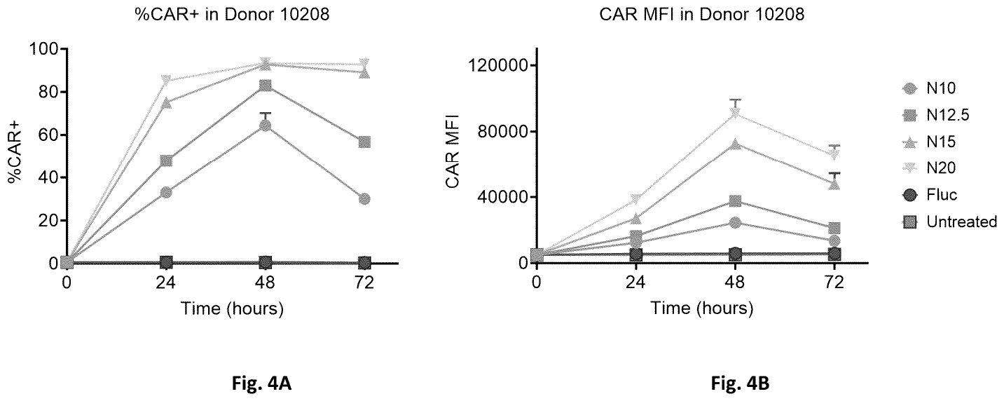

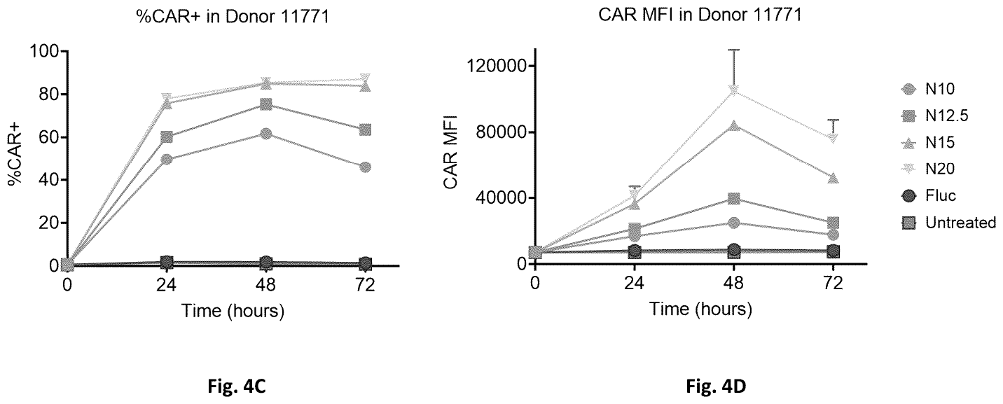

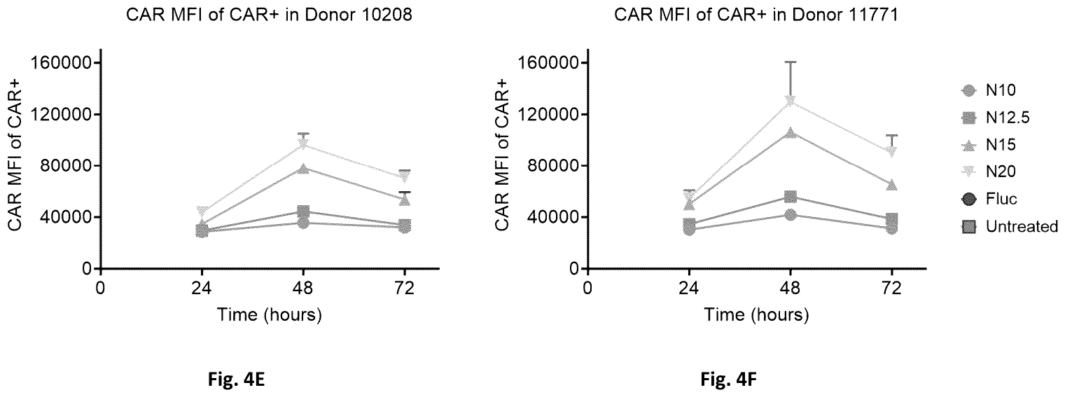

T cells from two different human donors (Donor 10208 and Donor 11771) were transfected with 0.11 ug/ml LNPs containing DSPC helper lipid at 10 mol %, 12.5 mol %, 15 mol %, and 20 mol %, respectively, and CAR mRNA and GFP mRNA. The percentage of CAR+ cells and CAR MFI were determined immediately or after 24, 48, 72, and 144 hours. T cells of both donors transfected with LNPs containing 15 mol % or 20 mol % DSPC demonstrated about 80% CAR+ cells over the 72 hour measurement period, while T cell transfected with LNPs containing 10 mol % or 12.5 mol % demonstrated lower percentages of CAR+ cells and the percentage of CAR+ T cells was reduced after 48 hours (FIGS. 4A and 4C). T cells of both donors transfected with LNPs containing 20 mol % DSPC demonstrated the highest CAR MFI, while CAR MFI was slightly lower in T cells transfected with LNPs containing 15 mol % and substantially lower in T cells transfected with LNPs containing 10 mol % and 12.5 mol % DSPC (FIGS. 4B and 4D).

Within CAR+ cells of both donors, T cells transfected with LNPs containing 20 mol % DSPC demonstrated the highest CAR MFI compared to T cells transfected with LNPs containing 10 mol %, 12.5 mol % or 15 mol % DSPC (FIGS. 4E and 4F).

These results demonstrated that LNPs containing 20 mol % DSPC helper lipid highly efficiently transfected human T cells of different donors and provided CAR expressing T cells over a period of 72 hours. In contrast, LNPs containing 10 mol % DSPC demonstrated the lowest transfection efficiency and resulted in loss of transfected at around 48 hours. LNPs containing 15 mol % DSPC demonstrated similar transfection efficiency to LNPs containing 20 mol % DSPC but lower overall CAR MFI. The performance of LNPs containing 12.5 mol % DSPC was intermediate between the 10 mol % DSPC LNPs and the 15 mol % LNPs.

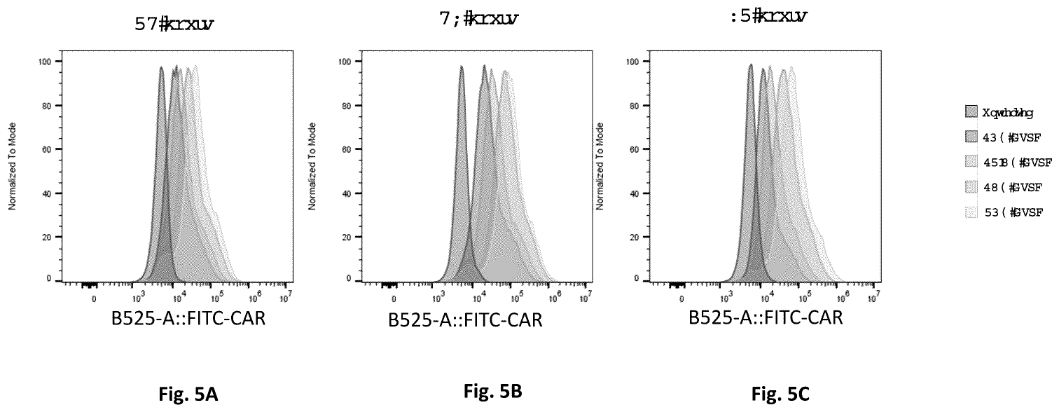

Binding and expression of transfected CARs to CAR-target protein was determined by a primary stain with recombinant antigen followed by a secondary stain with FITC- or APC-conjugated target antigen antibody and analysis by Flow Cytometry. T cells transfected with LNPs containing 20 mol % DSPC demonstrated the highest binding to immobilized CAR targets compared to T cells transfected with 15 mol %, 12.5 mol %, or 10 mol % DSPC LNPs (FIG. 5A-5C).

### Example 7: Transfection of Human Hematopoietic Stem Cells

CD117+/CD34+ human hematopoietic stem cells (HSCs) were used to generate humanized NSG™ mice. HSC-targeted LNPs coated with anti-CD117 Fab (Ab1) were engineered using LNP formulations comprising either 10 mol % DSPC (Formulation 1) or 20 mol % DSPC (Formulation 2) (see TABLE 2 for complete LNP compositions).

Humanized NSG™ mice were treated, by intravenous injection, with 1 mg/kg of LNP encapsulating mCherry mRNA coated with the anti-CD117 Fab and transfection efficiency was measured in NSG™ bone marrow.

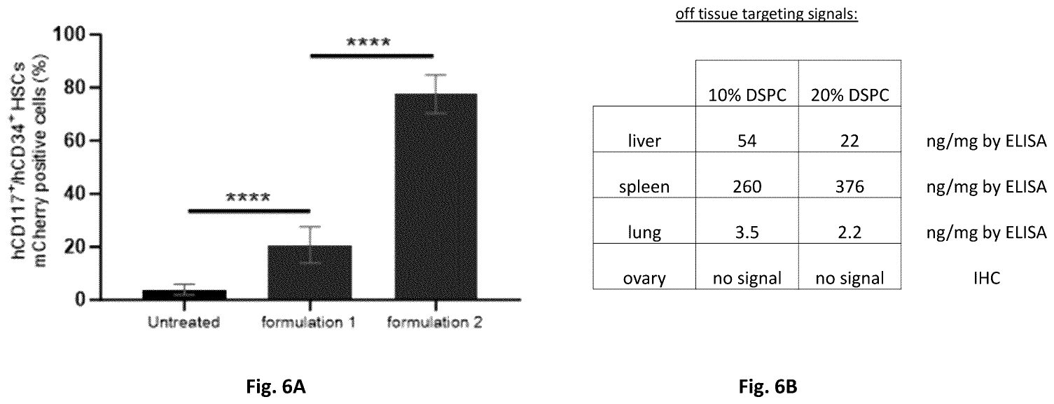

Using the two different LNP formulations, a range of transfection efficiency from 20% to about 80% was measured in vivo (FIG. 6A). Moreover, an increase over 2 folds of median fluorescent intensity of HSCs was measured in Formulation 2 transfected HSC compared to Formulation 1 transfected HSC in vivo (not shown). Off-target signals measured in liver and lung were lower in NSG™ mice treated with 20 mol % DSPC LNPs compared to 10 mol % DSPCs (FIG. 6B).

Overall, these results demonstrated the enhanced efficacy of LNPs described herein containing 20 mol % DSPC for transfection of human HSC in vivo.

CD117+/CD34+ human hematopoietic stem cells (HSCs) were cultured in vitro and transfected with varying doses of HSC-targeted LNPs coated with anti-CD117 Fab (Ab1)-using LNP formulations comprising either 10 mol % DSPC (Formulation 1) or 20 mol % DSPC (Formulation 2) (see TABLE 2 for complete LNP compositions).

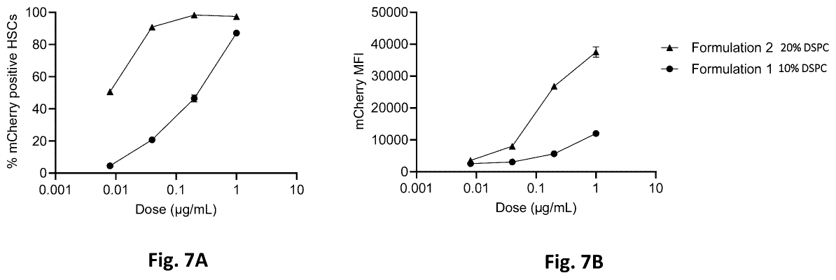

Using LNP Formulation 1 at the lowest dose of about 0.01 microgram/mL, the transfection efficiency was 5% whereas when using LNP Formulation 2 at the same dose the transfection efficiency was 50% (FIG. 7A). As seen in FIG. 7B, there was about 4-fold increase in MFI using LNP Formulation 2 at 1 microgram/mL, relative to LNP Formulation 1 at the same dose.

Overall, these results demonstrated the enhanced efficacy of LNPs described herein containing 20 mol % DSPC for transfection of human HSC in vitro.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various aspects of compositions of the present invention and/or in methods of the present invention, unless otherwise understood from the context. In other words, within this application, aspects have been described and depicted in a way that enables a clear and concise application to be written and drawn, but it is intended and will be appreciated that aspects may be variously combined or separated without parting from the present teachings and invention(s). For example, it will be appreciated that all features described and depicted herein can be applicable to all aspects of the invention(s) described and depicted herein.

### Embodiments

1. A lipid nanoparticle for targeted delivery of a nucleic acid into a target cell, the lipid nanoparticle comprising a cell-targeting group and a lipid blend comprising an ionizable lipid, a structural lipid, a helper lipid, and a PEG lipid, wherein the helper lipid is present in the lipid blend in a range of about 16 mol % to about 40 mol %.
2. The lipid nanoparticle of embodiment 1, wherein the ionizable lipid is a compound of formula

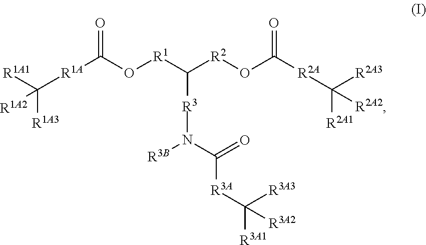

or a salt thereof, wherein:
   R¹, R², and R³ are each independently a bond or C₁₋₃ alkylene;
   R^{1A}, R^{2A}, and R^{3A} are each independently a bond or C₁₋₁₀ alkylene;
   R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀₀C(O) R^{a2};
   R^{a1} and R^{a2} are each independently C₁₋₂₀ alkyl or C₁₋₂₀ alkenyl; R^{3B} is

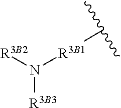

R^{3B1} is C₁₋₆ alkylene; and
R^{3B2} and R^{3B3} are each independently H or C₁₋₆ alkyl.
3. The lipid nanoparticle of embodiment 2, wherein the ionizable lipid is

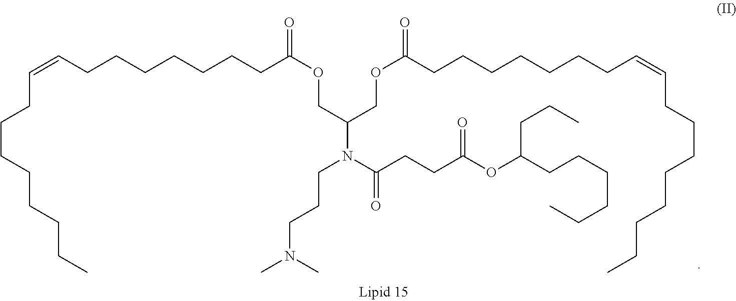 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

4. The lipid nanoparticle of any one of embodiments 1 to 3, further comprising a cargo.
5. The lipid nanoparticle of any one of embodiments 1 to 4, wherein the helper lipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.
6. The lipid nanoparticle of any one of embodiments 1 to 5, wherein the helper lipid is DSPC.
7. The lipid nanoparticle of any one of embodiments 1 to 6, wherein the helper lipid is present in the lipid blend in a range of about 16 mol % to about 30 mol %.
8. The lipid nanoparticle of embodiment 7, wherein the helper lipid is present in the lipid blend in a range of about 16 mol % to about 25 mol %.
9. The lipid nanoparticle of embodiment 8, wherein the helper lipid is present in the lipid blend at about 20 mol %.
10. The lipid nanoparticle of any one of embodiments 1 to 9, wherein the structural lipid is selected from the group consisting of cholesterol, fecosterol, β-sitosterol, ergosterol, campesterol, stigmasterol, stigmastanol, and brassicasterol.
11. The lipid nanoparticle of any one of embodiments 1 to 10, wherein the PEG lipid is selected from the group consisting of distearoylglycerol-PEG (DSG-PEG), distearoyl-phosphatidylethanolamine-PEG (DSPE-PEG), dimyrstoyl-phosphatidylethanolamine-PEG (DMPE-PEG), distearoyl-glycero-phosphoglycerol-PEG (DSPG-PEG), dimyristoyl-glycerol-PEG (DMG-PEG), dipalmitoyl-phosphatidylethanolamine-PEG (DPPE-PEG), dipalmitoyl-glycerol-PEG (DPG-PEG), and ceramide-PEG.
12. The lipid nanoparticle of any one of embodiments 1 to 11, wherein the ionizable lipid is present in the lipid blend in a range of about 30 mol % to about 70 mol %.
13. The lipid nanoparticle of any one of embodiments 1 to 12, wherein the ionizable lipid is present in the lipid blend in a range of about 40 mol % to about 60 mol %.
14. The lipid nanoparticle of any one of embodiments 1 to 13, wherein the ionizable lipid is present in the lipid blend in a range of about 48 mol % to about 50 mol %.
15. The lipid nanoparticle of any one of embodiments 1 to 14, wherein the structural lipid is present in the lipid blend in a range of about 20 mol % to about 50 mol %.
16. The lipid nanoparticle of any one of embodiments 1 to 15, wherein the structural lipid is present in the lipid blend in a range of about 30 mol % to about 40 mol %.
17. The lipid nanoparticle of any one of embodiments 1 to 16, wherein the structural lipid is cholesterol.
18. The lipid nanoparticle of embodiment 17, wherein cholesterol is present in the lipid blend in a range of about 25 mol % to about 40 mol %.
19. The lipid nanoparticle of any one of embodiments 1 to 18, wherein the PEG lipid is present in the lipid blend in a range of about 1 mol % to about 4 mol %.
20. The lipid nanoparticle of any one of embodiments 1 to 19, wherein the PEG lipid is DPG-PEG or DSPE-PEG.
21. The lipid nanoparticle of embodiment 20, wherein the PEG in DPG-PEG has a molecular weight of about 2000 daltons (DPG-PEG2K) and the PEG in DSPE-PEG has a molecular weight of about 2000 daltons (DSPE-PEG2K) or about 3400 daltons (DSPE-PEG3.4K).
22. The lipid nanoparticle of 21, wherein DPG-PEG2K, DSPE-PEG2K, or DSPE-PEG3.4K is present in the lipid blend in a range of about 1 mol % to about 2 mol %.
23. The lipid nanoparticle of any one of embodiments 1 to 22, wherein Lipid 15 is present in the lipid blend at about 49.25 mol %, cholesterol is present at about 29.25 mol %, DSPC is present at about 20 mol % and DPG-PEG2K is present at about 1.5 mol %.
24. The lipid nanoparticle of any one of embodiments 1 to 23, wherein the lipid nanoparticle has a mean diameter of about 60 nm to about 100 nm.
25. The lipid nanoparticle of any one of embodiments 1 to 24, wherein the lipid nanoparticle has a mean diameter of about 80 nm.
26. The lipid nanoparticle of any one of embodiments 1 to 25, wherein the lipid nanoparticle has a polydispersity index (PDI) of about 0.05 to about 1.
27. The lipid nanoparticle of embodiment 26, wherein the lipid nanoparticle has a PDI of about 0.09.
28. The lipid nanoparticle of any one of embodiments 1 to 27, wherein the lipid nanoparticle has a zeta potential (ZP) of about-30 mV to about +5 mV.
29. The lipid nanoparticle of embodiment 28, wherein the lipid nanoparticle has a ZP of about-1.4 mV.
30. The lipid nanoparticle of any one of embodiments 4 to 29, wherein the cargo comprises one or more nucleic acids.
31. The lipid nanoparticle of embodiment 30, wherein the one or more nucleic acids is a DNA.
32. The lipid nanoparticle of embodiment 30, wherein the one or more nucleic acid is an RNA.
33. The lipid nanoparticle of embodiment 32, wherein the RNA comprises a guide RNA and an RNA that encodes a Cas enzyme.
34. The lipid nanoparticle of any one of embodiments 30 to 32, wherein the one or more nucleic acid encodes a chimeric antigen receptor (CAR).
35. The lipid nanoparticle of any one of embodiments 1 to 34, wherein the cell-targeting group is coupled to a lipid of the lipid nanoparticle to form a lipid-cell targeting group conjugate.
36. The lipid nanoparticle of embodiment 35, wherein the cell-targeting group is coupled to the PEG-lipid.
37. The lipid nanoparticle of embodiment 36, wherein the cell-targeting group is coupled to a DPG-PEG2K, a DSPE-PEG2K or a DSPE-PEG3.4K.
38. The lipid nanoparticle of any one of embodiments 35 to 37, wherein the cell-targeting group comprises an antibody or fragment thereof.
39. The lipid nanoparticle of embodiment 38, wherein the cell-targeting group comprises an antibody.
40. The lipid nanoparticle of embodiment 38, wherein the cell-targeting group comprises a Fab fragment.
41. The lipid nanoparticle of embodiment 38, wherein the cell-targeting group comprises a single variable domain.
42. The lipid nanoparticle of any one of embodiments 35 to 41, wherein the cell-targeting group binds a molecule on an immune cell.
43. The lipid nanoparticle of embodiment 42, wherein the molecule on the immune cell is selected from the group consisting of CD3, CD4, CD7, and CD8.
44. The lipid nanoparticle of any one of embodiments 35 to 41, wherein the cell-targeting group binds a molecule on a hematopoietic stem cell.
45. The lipid nanoparticle of embodiment 44, wherein the molecule on the hematopoietic stem cell is selected from the group consisting of CD34, CD105, and CD117.
46. The lipid nanoparticle of any one of embodiments 35 to 45, wherein the lipid-cell targeting group conjugate is present in the lipid nanoparticle in a range of about 0.001 mol % to about 0.5 mol %.
47. A pharmaceutical composition comprising the lipid nanoparticle of any one of embodiments 1 to 46, and one or more pharmaceutically acceptable carriers or excipients.
48. A method of delivering a nucleic acid to a cell in a subject, the method comprising administering to the subject the lipid nanoparticle of any one of embodiments 1 to 46, or the pharmaceutical composition of embodiment 47.
49 The method of embodiment 48, wherein the cell is an immune cell.
50. The method of embodiment 48, wherein the cell is a hematopoietic stem cell.
51. Use of the lipid nanoparticle of any one of embodiments 1 to 46, or the pharmaceutical composition of embodiment 47 in the manufacture of a medicament for delivering a nucleic acid to a cell.
52. The use of embodiment 51, wherein the cell is an immune cell.
53. The use of embodiment 51, wherein the cell is a hematopoietic stem cell.
54. A lipid nanoparticle of any one of embodiments 1 to 46, or a pharmaceutical composition of embodiment 47 for use in delivering a nucleic acid to a cell.
55. The lipid nanoparticle of embodiment 54, wherein the cell is an immune cell.
56. The lipid nanoparticle of embodiment 54, wherein the cell is a hematopoietic stem cell.
57. A method of treating a disease in a subject in need thereof, the method comprising administering to the subject the lipid nanoparticle of any one of embodiments 1 to 46 or the pharmaceutical composition of embodiment 47.
58. A method of treating a disease in a subject, the method comprising administering to an immune cell of the subject in need thereof the lipid nanoparticle of any one of embodiments 1 to 46, or the pharmaceutical composition of embodiment 47.
59. The method of embodiment 58, wherein the administering is to a plurality of immune cells.
60. The method of embodiment 59, wherein the protein encoded by the one or more nucleic acids disposed in the lipid nanoparticle is detected in at least more than 60% (or even 80%) of immune cells at 72 hours after the administration of the lipid nanoparticle.
61. The method of embodiment 60, wherein the protein is detected in more immune cells in the subject at 72 hours after administration of the lipid nanoparticle, compared to another subject who received a lipid nanoparticle comprising the helper lipid that is present in the lipid blend at 10 mol %.
62. The method of embodiment 59, wherein the protein encoded by the one or more nucleic acids disposed in the lipid nanoparticle increases an effector function of the immune cell or plurality of immune cells compared to an immune cell or plurality of immune cells that are not administered the lipid nanoparticle.
63. A method of treating a disease in a subject, the method comprising administering to a hematopoietic stem cell of the subject in need thereof the lipid nanoparticle of any one of embodiments 1 to 41 and 44 to 46, or the pharmaceutical composition of embodiment 47.
64. The method of embodiment 63, wherein the lipid nanoparticle is administered to a plurality of hematopoietic stem cells of the subject.
65. The method of embodiment 63 or 64, wherein a protein encoded by the one or more nucleic acids disposed in the lipid nanoparticle is detected in at least 80% of hematopoietic stem cells of the plurality of hematopoietic stem cells of the subject.
66. The method of embodiment 65, wherein the protein is detected in about 20% of hematopoietic stem cells administered a lipid nanoparticle comprising the helper lipid that is present in the lipid blend at 10 mol %.
67. A lipid nanoparticle of any one of embodiments 1 to 46, or a pharmaceutical composition of embodiment 47 for use in the treatment of a disease in a subject in need thereof.
68. A lipid nanoparticle of any one of embodiments 1 to 43 and 46, or a pharmaceutical composition of embodiment 47 for use in the treatment of an immune cell-related disease in a subject in need thereof.
69. A lipid nanoparticle of any one of embodiments 1 to 41 and 44 to 46, or a pharmaceutical composition of embodiment 47 for use in the treatment of a hematopoietic stem cell-related disease in a subject in need thereof.
70. The lipid nanoparticle of embodiment 69, wherein the hematopoietic stem-cell related disease is sickle cell disease.

1. A lipid nanoparticle for targeted delivery of a nucleic acid into a target cell, the lipid nanoparticle comprising a cell-targeting group and a lipid blend comprising an ionizable lipid, a structural lipid, a helper lipid, and a PEG lipid, wherein the helper lipid is present in the lipid blend in a range of about 16 mol% to about 40 mol%.

2. The lipid nanoparticle of claim 1, wherein the ionizable lipid is a compound of formula I 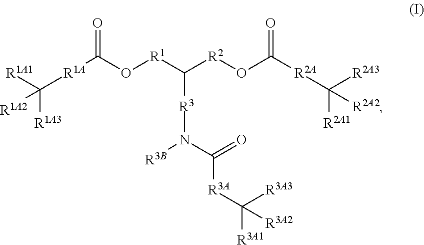
or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R3A3 are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl; R^{3B} is 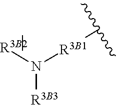
R^{3B1} is C₁₋₆ alkylene; and
R^{3B2} and R^{3B3} are each independently H or C₁₋₆ alkyl.

3. The lipid nanoparticle of claim 2, wherein the ionizable lipid is

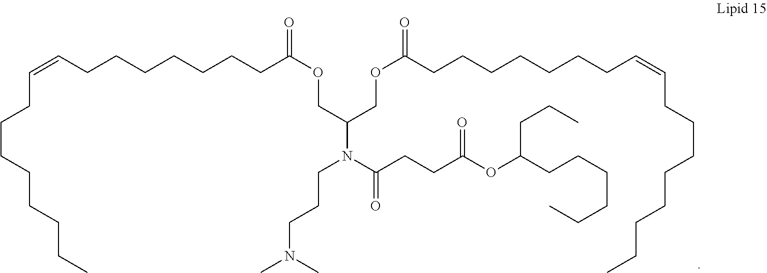 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

4. The lipid nanoparticle of claim 1, further comprising a cargo.

5. The lipid nanoparticle of claim 1, wherein:
(a) the helper lipid is selected from the group consisting of phosphatidylcholine, phosphatidylethanolamine, distearoyl-sn- glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero- 3-phosphocholine (DOPC), and sphingomyelin;
(b) the structural lipid is selected from the group consisting of cholesterol, fecosterol, β-sitosterol, ergosterol, campesterol, stigmasterol, stigmastanol, and brassicasterol; and/or
(c) the PEG lipid is selected from the group consisting of distearoylglycerol-PEG (DSG-PEG), distearoyl- phosphatidylethanolamine-PEG (DSPE-PEG), dimyrstoyl-phosphatidylethanolamine-PEG (DMPE-PEG), distearoyl-glycero-phosphoglycerol-PEG (DSPG-PEG), dimyristoyl-glycerol-PEG (DMG-PEG), dipalmitoyl- phosphatidylethanolamine-PEG (DPPE-PEG), dipalmitoyl-glycerol-PEG (DPG-PEG), and ceramide-PEG.

6. (canceled)

7. The lipid nanoparticle of claim 1, wherein the helper lipid is present in the lipid blend in a range of about 16 mol% to about 30 mol%.

8. The lipid nanoparticle of claim 7, wherein the helper lipid is present in the lipid blend in a range of about 16 mol% to about 25 mol% or wherein the helper lipid is present in the lipid blend at about 20 mol%.

9.- 11. (canceled)

12. The lipid nanoparticle of claim 1,, wherein:
(a) the ionizable lipid is present in the lipid blend in a range of about 30 mol% to about 70 mol%;
(b) the structural lipid is present in the lipid blend in a range of about 25 mol% to about 40 mol%;
(c) the PEG lipid is present in the lipid blend in a range of about 1 mol% to about 4 mol%;
(d) the helper lipid is present in the lipid blend in a range of about 16 mol% to about 30 mol%; and/or
(e) the PEG lipid is DPG-PEG or DSPE-PEG.

13.-17. (canceled)

18. The lipid nanoparticle of claim 1, wherein the PEG in DPG-PEG has a molecular weight of about 2000 daltons (DPG-PEG2K) and the PEG in DSPE-PEG has a molecular weight of about 2000 daltons (DSPE-PEG2K) or about 3400 daltons (DSPE-PEG3.4K).

19. The lipid nanoparticle of claim 3, wherein Lipid 15 is present in the lipid blend at about 49.25 mol%, cholesterol is present at about 29.25 mol%, DSPC is present at about 20 mol% and DPG-PEG2K is present at about 1.5 mol%.

20. The lipid nanoparticle of claim 1, wherein:
(a) the lipid nanoparticle has a mean diameter of about 60 nm to about 100 nm;
(b) the lipid nanoparticle has a polydispersity index (PDI) of about 0.05 to about 1; and/or
(c) the lipid nanoparticle has a zeta potential (ZP) of about −30 mV to about +5 mV.

21.- 22. (canceled)

23. The lipid nanoparticle of claim 4, wherein the cargo comprises one or more nucleic acids, optionally wherein the one or more nucleic acids is an RNA.

24. (canceled)

25. The lipid nanoparticle of claim 1, wherein the cell-targeting group is coupled to a lipid of the lipid nanoparticle to form a lipid-cell targeting group conjugate.

26. (canceled)

27. The lipid nanoparticle of claim 25, wherein the cell-targeting group comprises an antibody or fragment thereof.

28. The lipid nanoparticle of claim 25, wherein the cell-targeting group binds a molecule on an immune cell or wherein the cell-targeting group binds a molecule on a hematopoietic stem cell.

29. The lipid nanoparticle of claim 28, wherein the molecule on the immune cell is selected from the group consisting of CD3, CD4, CD7, and CD8 or wherein the molecule on the hematopoietic stem cell is selected from the group consisting of CD34, CD105, and CD117.

30.-31. (canceled)

32. The lipid nanoparticle of claim 25, wherein the lipid-cell targeting group conjugate is present in the lipid nanoparticle in a range of about 0.001 mol% to about 0.5 mol%.

33. A pharmaceutical composition comprising the lipid nanoparticle of claim 1.

34. A method of delivering a nucleic acid to a cell in a subject, the method comprising administering to the subject the lipid nanoparticle of claim 1.

35.-36. (canceled)

37. A method of treating a disease in a subject in need thereof, the method comprising administering to the subject the lipid nanoparticle of claim 1.
